(19) United States
(12) Patent Application Publication
(10) Pub. No.: US 2026/0034091 A1
(43) Pub. Date: Feb. 5, 2026

(54) Compositions and Methods for Treating Metabolic Disorders

(71) Applicant: The Regents of the University of California, Oakland, CA (US)

(72) Inventors: Anders Michael Näär, Berkeley, CA (US); Chi Zhu, Berkeley, CA (US); Justin Yu-Sun Lee, Berkeley, CA (US)

(21) Appl. No.: 19/099,290

(22) PCT Filed: Jul. 27, 2023
(86) PCT No.: PCT/US2023/028833

§ 371 Date: Jan. 28, 2025

Related U.S. Application Data
(60) Provisional application No. 63/393,585, filed on Jul. 29, 2022.

(51) Int. Cl. A61K 31/341; A61K 38/28; A61K 45/06; A61P 1/16; A61P 3/04

(52) U.S. Cl. CPC A61K 31/341; A61K 38/28; A61K 45/06; A61P 1/16; A61P 3/04

(57) ABSTRACT

The present disclosure provides methods for treating metabolic disorders. The methods generally involve administering to an individual in need thereof an effective amount of a compound of Formula I.

### CROSS-REFERENCE

This application claims the benefit of U.S. Provisional Patent Application No. 63/393,585, filed Jul. 29, 2022, which application is incorporated herein by reference in its entirety.

### INTRODUCTION

Nonalcoholic fatty liver disease (NAFLD) has emerged as a major source of liver disease globally. Clinically, NAFLD describes a spectrum of hepatic events ranging from moderate lipid accumulation to more aggressive steatosis with associated inflammation, ballooning hepatocytes, fibrosis, cirrhosis, and, in some cases, hepatocellular carcinoma (HCC). The excessive accumulation of lipids is a major risk factor for disease progression from the clinically silent NAFLD to the inflammatory, fibrotic, and cirrhotic nonalcoholic steatohepatitis (NASH) stage.

Metabolic disorders such as insulin resistance, hyperglycemia, type 2 diabetes mellitus, obesity, fatty liver disease, glucose intolerance, hyperinsulinemia, metabolic syndrome, and hypertension, are major health problems.

There is a need in the art for methods of treating metabolic disorders.

### SUMMARY

The present disclosure provides methods for treating metabolic disorders. The methods generally involve administering to an individual in need thereof an effective amount of a compound of Formula I.

### BRIEF DESCRIPTION OF THE DRAWINGS

FIG. 1A-1C illustrates the dose dependent effect of TOFA treatment in mice with CDAHFD induced NAFLD progression.

FIG. 2A-21 illustrate the effect of TOFA treatment on 60% kcal fat diet induced metabolic syndrome and early stage NAFLD progression.

FIG. 3A-3F show the rescue effect of TOFA treatment CDAHFD induced late stage NAFLD/NASH progression in mice.

FIG. 4A-4E show the results of RNA sequencing of liver samples from mice treated with TOFA from CDAHFD-induced late stage NAFLD/NASH.

FIG. 5A-5G illustrate the safety and tolerability profile of TOFA treatment in ad libitum fed chow mice.

FIG. 6A-6H illustrate the dose dependent effect of TOFA treatment in mice with diet-induced obesity with a 60% HFD dietary model.

FIG. 7A-7D show a transcriptional analysis of genes changed in the liver from TOFA treatment in a 60% HFD dietary-induced obesity mouse model.

FIG. 8A-8I show an in-depth analysis of TOFA treatment efficacy in a CDAHFD dietary-induced mouse model of NAFLD/NASH.

FIG. 9A-9I show a benchmark efficacy comparison of TOFA compared to a vehicle control, Firsocostat, Fenofibrate, and a combination treatment of Firsocostat and Fenofibrate.

FIG. 10A-10I show a treatment efficacy study of TOFA and Semaglutide as single agent therapeutics and in combination.

FIG. 11A-11P show TOFA action in a genetic PPARA knockout model for both a CDAHFD and 60% kcal fat (HFD) dietary-induced model of NAFLD/NASH and DIO, respectively.

### DEFINITIONS

The terms “diabetes” and “diabetic” refer to a progressive disease of carbohydrate metabolism involving inadequate production or utilization of insulin, frequently characterized by hyperglycemia and glycosuria. The terms “pre-diabetes” and “pre-diabetic” refer to a state wherein a subject does not have the characteristics, symptoms and the like typically observed in diabetes, but does have characteristics, symptoms and the like that, if left untreated, may progress to diabetes. The presence of these conditions may be determined using, for example, either the fasting plasma glucose (FPG) test or the oral glucose tolerance test (OGTT). Both usually require a subject to fast for at least 8 hours prior to initiating the test. In the FPG test, a subject's blood glucose is measured after the conclusion of the fasting; generally, the subject fasts overnight and the blood glucose is measured in the morning before the subject eats. A healthy subject would generally have a FPG concentration between about 90 and about 100 mg/dl, a subject with “pre-diabetes” would generally have a FPG concentration between about 100 and about 125 mg/dl, and a subject with “diabetes” would generally have a FPG level above about 126 mg/dl. In the OGTT, a subject's blood glucose is measured after fasting and again two hours after drinking a glucose-rich beverage. Two hours after consumption of the glucose-rich beverage, a healthy subject generally has a blood glucose concentration below about 140 mg/dl, a pre-diabetic subject generally has a blood glucose concentration about 140 to about 199 mg/dl, and a diabetic subject generally has a blood glucose concentration about 200 mg/dl or above. While the aforementioned glycemic values pertain to human subjects, normoglycemia, moderate hyperglycemia and overt hyperglycemia are scaled differently in murine subjects. A healthy murine subject after a four-hour fast would generally have a FPG concentration between about 100 and about 150 mg/dl, a murine subject with “pre-diabetes” would generally have a FPG concentration between about 175 and about 250 mg/dl and a murine subject with “diabetes” would generally have a FPG concentration above about 250 mg/dl.

The term “insulin resistance” as used herein refers to a condition where a normal amount of insulin is unable to produce a normal physiological or molecular response. In some cases, a hyper-physiological amount of insulin, either endogenously produced or exogenously administered, is able to overcome the insulin resistance, in whole or in part, and produce a biologic response.

The term “metabolic syndrome” refers to an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of Type 2 diabetes and/or other disorders (e.g., atherosclerosis).

The term “glucose metabolism disorder” encompasses any disorder characterized by a clinical symptom or a combination of clinical symptoms that is associated with an elevated level of glucose and/or an elevated level of insulin in a subject relative to a healthy individual. Elevated levels of glucose and/or insulin may be manifested in the following diseases, disorders and conditions: hyperglycemia, type 2 diabetes, gestational diabetes, type 1 diabetes, insulin resistance, impaired glucose tolerance, hyperinsulinemia, impaired glucose metabolism, pre-diabetes, other metabolic disorders (such as metabolic syndrome, which is also referred to as syndrome X), and obesity, among others. The polypeptides of the present disclosure, and compositions thereof, can be used, for example, to achieve and/or maintain glucose homeostasis, e.g., to reduce glucose level in the bloodstream and/or to reduce insulin level to a range found in a healthy subject.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “hyperinsulinemia”, as used herein, refers to a condition in which there are elevated levels of circulating insulin when, concomitantly, blood glucose levels are either elevated or normal. Hyperinsulinemia can be caused by insulin resistance which is associated with dyslipidemia, such as high triglycerides, high cholesterol, high low-density lipoprotein (LDL) and low high-density lipoprotein (HDL); high uric acids levels; polycystic ovary syndrome; type 2 diabetes and obesity. Hyperinsulinemia can be diagnosed as having a plasma insulin level higher than about 2 μU/mL.

As used herein, the phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters squared. An adult having a BMI in the range of from about 18.5 kg/m² to about 24.9 kg/m² is considered to have a normal weight; an adult having a BMI between about 25 kg/m² and about. 29.9 kg/m² may be considered overweight (pre-obese); and an adult having a BMI of about 30 kg/m² or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets. In some cases, an “individual” is a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a compound of Formula I” includes a plurality of such compounds and reference to “the metabolic disorder” includes reference to one or more metabolic disorder and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides method for treating metabolic disorders. The methods generally involve administering to an individual in need thereof an effective amount of a compound of Formula I.

### TOFA and TOFA Analogs

In some cases, the present disclosure provides a method of treating a metabolic disorder in an individual, the method comprising administering to the patient a therapeutically effective amount of 5-(Tetradecyloxy)-2-furoic acid (TOFA) or a TOFA analog or derivative. TOFA has the following structure:

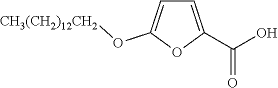

In some cases, the present disclosure provides a method of treating a metabolic disorder in an individual, the method comprising administering to the patient a therapeutically effective amount of an analog of TOFA. For example, in some cases, a method of the present disclosure comprises administering a compound of Formula I:

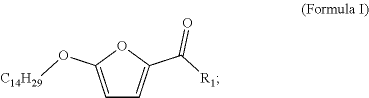

wherein: R¹ is R¹ is —O—R², —O—R³—OR², —O—R³—OC(O)—N(R⁵)R⁶, —O—R³—N(R⁵)R⁶, —O—R³—N(R⁴)C(O)OR⁵, —O—R³—C(O)OR⁵, —O—R³—C(O)N(R⁵)R⁶ or —N(R⁵)S(O)₂—R⁴;
each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; each R³ is independently an optionally substituted alkylene chain; R⁴ is optionally substituted alkyl, optionally substituted aryl, optionally substituted aralkyl, optionally substituted heteroaryl or optionally substituted heteroarylalkyl; each R⁵ is independently hydrogen, alkyl, optionally substituted cycloalkyl, optionally substituted aryl or optionally substituted aralkyl; each R⁶ is alkyl, optionally substituted cycloalkyl, optionally substituted aralkyl or —R³C(O)OR⁴ ; or any R⁵ and R⁶, together with the nitrogen to which they are both attached, form an optionally substituted N-heterocyclyl or an optionally substituted N-heteroaryl; as a single stereoisomer or as a mixture thereof or a pharmaceutically acceptable salt thereof.

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, unless specified to the contrary, the following terms have the meaning indicated: “Amino” refers to the —NH₂ radical. “Cyano” refers to the —CN radical. “Hydroxy” refers to the —OH radical. “Imino” refers to the ═NH substituent. “Nitro” refers to the —NO₂ radical. “Oxo” refers to the ═O substituent. “Thioxo” refers to the ═S substituent. “Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O), R¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, alkenyl, halo, haloalkyl, haloalkenyl, cyano, nitro, aryl, aralkyl, heteroaryl, heteroarylalkyl, —R¹⁵—OR¹⁴,—R¹⁵—OC(O)—R¹⁴, —R¹⁵—N(R¹⁴)₂, —R¹⁵—C(O)R¹⁴, —R¹⁵—C(O)OR¹⁴,—R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN (R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and,—R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl [1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxo-1,3-dioxol-4yl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)OR¹⁴, —R¹⁵—C(O)OR¹⁴, —R¹⁵—C(O)N(R¹⁴)₂, —R¹⁵—N(R¹⁴)C(O)OR¹⁶, —R¹⁵ —N(R¹⁴)C(O)R¹⁶, —R¹⁵ —N(R¹⁴)S(O)ₜR¹⁶ (where t is 1 to 2), —R¹⁵—N═C(OR¹⁴)R¹⁴, —R¹⁵—(S(O)ₜOR¹⁶ (where t is 1 to 2), —R¹⁵—S(O)ₚR¹⁶ (where p is 0 to 2), and —R¹⁵—S(O)ₜN(R¹⁴)₂(where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkylene chain at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e, thienyl). Unless stated otherwise specifically in the specification, the term “heteroaryl” is meant to include heteroaryl radicals as defined above which are optionally substituted by one or more substituents selected from the group consisting of alkyl, alkenyl, alkoxy, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substituents on the functional group are also “optionally substituted” and so on, for the purposes of this invention, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Examples of inorganic salts include the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Examples of organic bases include isopropylamine, diethylamine, ethanolamine, trimethylamine, dicyclohexylamine, choline and caffeine.

A “pharmaceutical composition” refers to a formulation of a compound and a medium generally accepted in the art for the delivery of the biologically active compound to a mammal, e.g., humans. Such a medium can include a pharmaceutically acceptable carrier, diluent, or excipient.

“Therapeutically effective amount” refers to that amount of a compound which, when administered to a mammal, e.g., a human, is sufficient to effect treatment of the disease or condition of interest in a mammal, e.g., a human, having the disease or condition. The amount of a compound which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure.

A compound as disclosed herein, or a pharmaceutically acceptable salt thereof, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. A compound as disclosed herein is meant to include all such possible isomers, as well as their racemic and optically pure forms. Optically active (+) and (−), (R)- and(S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

The use of parentheses in substituent groups is used herein to conserve space. Accordingly, the use of parenthesis in a substituent group indicates that the group enclosed within the parentheses is attached directly to the atom preceding the parenthesis.

Of the compounds of formula (I), one embodiment is a compound of formula (I) wherein: R¹ is —O—R²; and R² is independently alkyl or heterocyclylalkyl. Of this embodiment, one embodiment is a compound of formula (I) selected from: isopropyl 5-(tetradecyloxy) furan-2-carboxylate; 4-methylpentyl 5-(tetradecyloxy)furan-2-carboxylate; and (5-methyl-2-oxo-1,3-dioxol-4-yl)methyl 5-(tetradecyloxy)furan-2-carboxylate.

Of the compounds of formula (I), another embodiment is a compound of formula (I) wherein: R¹ is —O—R²; and R² is haloalkyl or substituted aryl. Of this embodiment, one embodiment is a compound of formula (I) selected from: 2,2,2-trifluoroethyl 5-(tetradecyloxy)furan-2-carboxylate; 2,2,2-trichloroethyl 5-(tetradecyloxy)furan-2-carboxylate; 2-bromoethyl 5-(tetradecyloxy)furan-2-carboxylate; and 2-(5-(tetradecyloxy)furan-2-carbonyloxy)benzoic acid.

Of the compounds of formula (I), another embodiment is a compound of formula (I) wherein: R¹ is —O—R³—OR²; R² is optionally substituted heterocyclylalkyl; and R³ is an optionally substituted alkylene chain. Of this embodiment, one embodiment is a compound of formula (I) which is 3-(tetrahydro-2H-pyran-2-yloxy)propyl 5-(tetradecyloxy)furan-2-carboxylate.

Of the compounds of formula (I), another embodiment is a compound of formula (I) wherein: R¹ is —O—R³—OC(O)-N(R⁵)R⁶; each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; R³ is an optionally substituted alkylene chain; and R⁵ is hydrogen, alkyl, optionally substituted cycloalkyl, optionally substituted aryl or optionally substituted aralkyl; and R⁶ is alkyl, optionally substituted cycloalkyl, optionally substituted aralkyl or —R³C(O)OR³; and/or any R⁵ and R⁶, together with the nitrogen to which they are both attached, form an optionally substituted N-heterocyclyl or an optionally substituted N-heteroaryl. Of this embodiment, one embodiment is a compound of formula (I) selected from: 1-(benzyl(methyl)carbamoyloxy)ethyl 5-(tetradecyloxy)furan-2-carboxylate; 1-((2-ethoxy-2-oxoethyl)(methyl)carbamoyloxy)ethyl 5-(tetradecyloxy)furan-2-carboxylate; 4-(2S)-2-benzyl 1-(1-(5-(tetradecyloxy)furan-2-carbonyloxy)ethyl)pyrrolidine-1,2-dicarboxylate; 1-(4-phenylcyclohexanecarbonyloxy)ethyl 5-(tetradecyloxy)furan-2-carboxylate; 1-(5-(tetradecyloxy)furan-2-carbonyloxy)ethyl 3-phenylpyrrolidine-1-carboxylate; 1-(5-(tetradecyloxy)furan-2-carbonyloxy)ethyl 3,4-dihydroisoquinoline-2(1H)-carboxylate: 1-(5-(tetradecyloxy)furan-2-carbonyloxy)ethyl piperidine-1-carboxylate; 1-(5-(tetradecyloxy)furan-2-carbonyloxy)ethyl morpholine-4-carboxylate; 1-tert-butyl 4-(1-(5-(tetradeclyoxy)furan-2-carbonyloxy)ethyl)piperazine-1,4-dicarboxylate; and 1-(dicyclohexylcarbamoyloxy)ethyl 5-(tetradecyloxy)furan-2-carboxylate.

Of the compounds of formula (I), another embodiment is a compound of formula (I) wherein: R¹ is —O—R³—N(R⁵)R⁶; R³ is an optionally substituted alkylene chain; and R⁵ is hydrogen, alkyl, optionally substituted cycloalkyl, optionally substituted aryl or optionally substituted aralkyl; and R⁶ is alkyl, optionally substituted cycloalkyl, optionally substituted aralkyl or —R³—C(O)OR⁴ ; and/or any R⁵ and R⁶, together with the nitrogen to which they are both attached, form an optionally substituted N-heterocyclyl or an optionally substituted N-heteroaryl. Of this embodiment, one embodiment is a compound of formula (I) selected from: 2-(dimethylamino)ethyl 5-(tetradecyloxy)furan-2-carboxylate; 2-morpholinoethyl 5-(tetradecyloxy)furan-2-carboxylate; or 3-morpholinopropyl 5-(tetradecyloxy)furan-2-carboxylate.

Of the compounds of formula (I), another embodiment is a compound of formula (I) wherein: R¹ is —O—R³—N(R⁴ )C(O)OR⁵; R³ is an optionally substituted alkylene chain; and R⁴ is optionally substituted alkyl, optionally substituted aryl, optionally substituted aralkyl, optionally substituted heteroaryl or optionally substituted heteroarylalkyl; and R⁵ is hydrogen, alkyl, optionally substituted cycloalkyl, optionally substituted aryl or optionally substituted aralkyl.

Of the compounds of formula (I), another embodiment is a compound of formula (I) wherein: R¹ is —O—R³—C(O)OR⁵; R³ is an optionally substituted alkylene chain; and R⁵ is hydrogen, alkyl, optionally substituted cycloalkyl, optionally substituted aryl or optionally substituted aralkyl.

Of the compounds of formula (I), another embodiment is a compound of formula (I) wherein: R¹ is —O—R³—C(O)N(R³)R⁶; R³ is an optionally substituted alkylene chain; and R⁵ is hydrogen, alkyl, optionally substituted cycloalkyl, optionally substituted aryl or optionally substituted aralkyl; and R⁶ is alkyl, optionally substituted cycloalkyl, optionally substituted aralkyl or R³C(O)OR⁴ ; or R⁵ and R⁶, together with the nitrogen to which they are both attached, form an optionally substituted N-heterocyclyl or an optionally substituted N-heteroaryl. Of this embodiment, one embodiment is a compound of formula (I) selected from: 2-(benzyl(methyl)amino)-2-oxoethyl 5-(tetradecyloxy)furan-2-carboxylate; tert-butyl 4-(2-(5-tetradecyloxy)furan-2-carbonyloxy) acetyl)piperazine-1-carboxylate; 2-(dicyclohexylamino)-2-oxoethyl 5-(tetradecyloxy)furan-2-carboxylate; 2-(4-cyclohexylpiperazin-1-yl)-2-oxoethyl 5-(tetradecyloxy)furan-2-carboxylate; 2-oxo-2-(4-phenylpiperzin-1-yl)ethyl-5-(tetradecyloxy)furan-2-carboxylate; 2-((2-ethoxy-2-oxoethyl)(methyl)amino)-2-oxoethyl 5-tetradecyloxy)furan-2-carboxylate; 2-oxo-2-(piperidin-1-yl)ethyl-5-(tetradecyloxy)furan-2-carboxylate; 2-morpholino-2-oxoethyl 5-(tetradecyloxy)furan-2-carboxylate; 2-(3,4-dihydroisoquinolin-2(1H)-yl)-2-oxoethyl 5-(tetradecyloxy)furan-2-carboxylate; and (S)-benzyl 1-(2-(5-(tetradecyloxy)furan-2-carbonyloxy)acetyl)pyrrolidine-2-carboxylate.

Of the compounds of formula (I), another embodiment is a compound of formula (I) wherein: R¹ is —N(R⁵)S(O)₂—R⁴; R⁴ is optionally substituted alkyl, optionally substituted aryl, optionally substituted aralkyl, optionally substituted heteroaryl or optionally substituted heteroarylalkyl; and R⁵ is independently hydrogen, alkyl, optionally substituted cycloalkyl, optionally substituted aryl or optionally substituted aralkyl. Of this embodiment, one embodiment is a compound of formula (I) which is 5-(tetradecyloxy)-N-tosylfuran-2-carboxamide.

Of the pharmaceutical compositions, one embodiment is wherein the pharmaceutical composition is an oral composition comprising an effective amount of a compound of formula (I), or a pharmaceutically acceptable salt thereof.

### Pharmaceutical Compositions

A compound of Formula I, or a pharmaceutically acceptable salt thereof, can be in the form of compositions suitable for administration to a subject. In general, such compositions are “pharmaceutical compositions” comprising a compound of Formula I (or a pharmaceutically acceptable salt thereof) and one or more pharmaceutically acceptable or physiologically acceptable diluents, carriers or excipients. In some cases, a compound of Formula I (or a pharmaceutically acceptable salt thereof) is present in a therapeutically effective amount. The pharmaceutical compositions may be used in the methods of the present disclosure; thus, for example, the pharmaceutical compositions can be administered to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds (e.g., glucose lowering agents) as described herein in order to treat or prevent the diseases, disorders and conditions as contemplated by the present disclosure.

The pharmaceutical compositions typically comprise a therapeutically effective amount of a compound of Formula I (or a pharmaceutically acceptable salt thereof) and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that could be used in the pharmaceutical compositions and dosage forms. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino) ethanesulfonic acid sodium salt (MES). 3-(N-Morpholino) propanesulfonic acid (MOPS), and N-Tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen™)), whereas a multi-use container (e.g., a multi-use vial) is provided in other embodiments. Any drug delivery apparatus may be used to deliver a compound of Formula I (or a pharmaceutically acceptable salt thereof), including implants (e.g., implantable pumps) and catheter systems, both of which are well known to the skilled artisan. Depot injections, which are generally administered subcutaneously or intramuscularly, may also be utilized to release the polypeptides disclosed herein over a defined period of time. Depot injections are usually either solid-or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

The pharmaceutical compositions containing the active ingredient (e.g., a compound of Formula I (or a pharmaceutically acceptable salt thereof)) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods of preparing liposomes are described in, for example, U.S. Pat. Nos. 4,235,871, 4,501,728, and 4,837.028. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

In some cases, a compound of Formula I (or a pharmaceutically acceptable salt thereof) is not formulated for topical administration.

### Methods

A method of the present disclosure comprises administering to an individual in need thereof a therapeutically effective amount of a compound of Formula I, or a pharmaceutically acceptable salt thereof.

A “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects. In some cases, e.g., in the case of a hyperglycemic condition, a lowering or reduction of blood glucose or an improvement in glucose tolerance test can be used to determine whether the amount of an agent is effective to treat the hyperglycemic condition. The therapeutically effective amount can be adjusted in connection with the dosing regimen and diagnostic analysis of the subject's condition and the like. A “compound of Formula I” is meant to encompass pharmaceutically acceptable salts of a compound of Formula I, unless specifically stated otherwise.

In some cases, a therapeutically effective amount of a compound of Formula I is an amount that, when administered in one or more doses, is sufficient to reduce or decrease any level (e.g., a baseline level) of fasting plasma glucose (FPG), wherein, for example, the amount is sufficient to reduce a FPG level greater than 200 mg/dl to less than 200 mg/dl, wherein the amount is sufficient to reduce a FPG level between 175 mg/dl and 200 mg/dl to less than the starting level, wherein the amount is sufficient to reduce a FPG level between 150 mg/dl and 175 mg/dl to less than the starting level, wherein the amount is sufficient to reduce a FPG level between 125 mg/dl and 150 mg/dl to less than the starting level, and so on (e.g., reducing FPG levels to less than 125 mg/dl, to less than 120 mg/dl, to less than 115 mg/dl, to less than 110 mg/dl, etc.).

In some cases, a therapeutically effective amount of a compound of Formula I is an amount is an amount that, when administered in one or more doses, is sufficient to reduce or decrease hemoglobin A1c (HbA1c) levels by more than about 10% to 9%, by more than about 9% to 8%, by more than about 8% to 7%, by more than about 7% to 6%, by more than about 6% to 5%, and so on. In some cases, a therapeutically effective amount of a compound of Formula I is an amount is an amount sufficient to reduce or decrease HbA1c levels by about 0.1%, 0.25%, 0.4%, 0.5%, 0.6%, 0.7%, 0.8%, 0.9%, 1%, 1.5%, 2%, 3%, 4%, 5%, 10%, 20%, 30%, 33%, 35%, 40%, 45%, 50%, or more.

In some cases, a therapeutically effective amount of a compound of Formula I is an amount is an amount that, when administered in one or more doses, is sufficient to result in insulin levels in a normal range.

In some cases, a therapeutically effective amount of a compound of Formula I is an amount is an amount that, when administered in one or more doses, is sufficient to result in serum alanine transaminase (ALT) levels in a normal range. In some cases, a therapeutically effective amount of a compound of Formula I is an amount is an amount that, when administered in one or more doses, is sufficient to reduce serum ALT levels by at least at least about 10%, at least about 20%, at least about 25%, at least about 30%, at least about 40%, at least about 50%, or more than 50%, compared to the serum ALT level before treatment. In some cases, a therapeutically effective amount of a compound of Formula I is an amount is an amount that, when administered in one or more doses, is sufficient to result in serum aspartate transaminase (AST) in a normal range. In some cases, a therapeutically effective amount of a compound of Formula I is an amount is an amount that, when administered in one or more doses, is sufficient to reduce serum AST levels by at least at least about 10%, at least about 20%, at least about 25%, at least about 30%, at least about 40%, at least about 50%, or more than 50%, compared to the serum AST level before treatment.

In some cases, an effective amount of a compound of Formula I is an amount that, when administered in one or more doses to a subject, produces a desired result relative to a healthy subject. For example, an effective dose may be one that, when administered to a subject having elevated plasma glucose and/or plasma insulin, achieves a desired reduction relative to that of a healthy subject by at least about 10%, at least about 20%, at least about 25%, at least about 30%, at least about 40%, at least about 50%, at least about 60%, at least about 70%, at least about 80%, or more than 80%.

### Routes of Administration

Suitable routes of administration include oral, rectal, nasal, pulmonary, topical, subcutaneous, intramuscular, intraperitoneal, intravenous, intradermal, intrathecal, and epidural. In some cases, the route of administration is oral.

### Dosages

A compound of Formula I may be administered to a subject in an amount that is dependent upon, for example, the goal of the administration (e.g., the degree of resolution desired); the age, weight, sex, and health and physical condition of the subject to be treated; the nature of the compound, and/or formulation being administered; the route of administration; and the nature of the disease, disorder, condition or symptom thereof (e.g., the severity of the dysregulation of glucose/insulin and the stage of the disorder). The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with absorption, distribution. metabolism, and excretion (“ADME”), taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

An appropriate dosage level will generally be about 0.001 to 100 mg/kg of patient body weight per day, which can be administered in single or multiple doses.

In some cases, the dosage level will be about 0.01 to about 25 mg/kg per day, and in other embodiments about 0.05 to about 10 mg/kg per day. A suitable dosage level may be about 0.01 to 25 mg/kg per day, about 0.05 to 10 mg/kg per day, or about 0.1 to 5 mg/kg per day. Within this range, the dosage may be 0.005 to 0.05, 0.05 to 0.5 or 0.5 to 5.0 mg/kg per day.

In some cases, a therapeutically effective amount of a compound of Formula (I), a pharmaceutically acceptable salt, solvate, polymorph, prodrug, metabolite, stereoisomer, or isomer thereof is about 25 mg per day, about 50 mg per day, about 75 mg per day, about 100 mg per day, about 150 mg per day, about 200 mg per day, or about 400 mg per day. In some embodiments, the therapeutically effective amount of a compound of Formula (I), a pharmaceutically acceptable salt, solvate, polymorph, prodrug, metabolite, stereoisomer, or isomer thereof is about 50 mg per day, about 100 mg per day, about 150 mg per day, about 200 mg per day, or about 400 mg per day. In some embodiments, the therapeutically effective amount of a compound of Formula (I), a pharmaceutically acceptable salt, solvate, polymorph, prodrug, metabolite, stereoisomer, or isomer thereof is up to 25 mg per day, up to 50 mg per day, up to 75 mg per day, up to 100 mg per day, up to 150 mg per day, up to 200 mg per day, up to 400 mg per day, up to 600 mg per day, up to 800 mg per day, or up to 1000 mg per day. In some embodiments, the therapeutically effective amount of a compound of Formula (I), a pharmaceutically acceptable salt, solvate, polymorph, prodrug, metabolite, N-oxide, stereoisomer, or isomer thereof is up to 400 mg per day

For administration of an oral agent, the compositions can be provided in the form of tablets, capsules and the like containing from 1.0 to 1000 milligrams of the active ingredient, particularly 1.0. 3.0. 5.0, 10.0. 15.0, 20.0, 25.0, 50.0, 75.0, 100.0, 150.0. 200.0, 250.0. 300.0, 400.0, 500.0, 600.0, 750.0. 800.0, 900.0, and 1000.0 milligrams of the active ingredient. A compound of Formula I may be administered on a regimen of, for example, 1 to 4 times per day, and often once or twice per day.

The dosage of a compound of Formula I may be repeated at an appropriate frequency, which may be in the range of once per day to once every three months, depending on the pharmacokinetics of the compound (e.g., half-life) and the pharmacodynamic response (e.g., the duration of the therapeutic effect of the compound. In some cases, dosing is frequently repeated between once per week and once every 3 months. In other instances, a compound of Formula I is administered approximately once per month.

In certain embodiments, the dosage of a compound of Formula I is contained in a “unit dosage form”. The phrase “unit dosage form” refers to physically discrete units, each unit containing a predetermined amount of a compound of Formula I, either alone or in combination with one or more additional agents, sufficient to produce the desired effect. It will be appreciated that the parameters of a unit dosage form will depend on the particular agent and the effect to be achieved.

### Combination Therapy

The present disclosure contemplates the use of a compound of Formula I in combination with one or more additional agents (e.g., one or more additional active therapeutic agents) or other prophylactic or therapeutic modalities. In such combination therapy, the various active agents frequently have different mechanisms of action. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain cases, a compound of Formula I and the at least one additional agent are administered or applied sequentially, e.g., where one agent is administered prior to one or more other agents. In other cases, a compound of Formula I and the at least one additional agent are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

A compound of Formula I can be used in combination with other agents useful in the treatment of the disorders or conditions set forth herein, including those that are normally administered to subjects suffering from obesity, eating disorder, hyperglycemia, hyperinsulinemia, glucose intolerance, and other glucose metabolism disorders.

The present disclosure contemplates combination therapy with numerous agents (and classes thereof), including 1) insulin, insulin mimetics and agents that entail stimulation of insulin secretion, including sulfonylureas (e.g., chlorpropamide, tolazamide, acetohexamide, tolbutamide, glyburide, glimepiride, glipizide) and meglitinides (e.g., mitiglinide, repaglinide and nateglinide); 2) biguanides (e.g., metformin, and its pharmaceutically acceptable salts, in particular, metformin hydrochloride, and extended-release formulations thereof, such as Glumetza™, Fortamet™, and GlucophageXR™) and other agents that act by promoting glucose utilization, reducing hepatic glucose production and/or diminishing intestinal glucose output; 3) alpha-glucosidase inhibitors (e.g., acarbose, voglibose and miglitol) and other agents that slow down carbohydrate digestion and consequently absorption from the gut and reduce postprandial hyperglycemia; 4) thiazolidinediones (e.g., rosiglitazone, troglitazone, pioglitazone, glipizide, balaglitazone, rivoglitazone, netoglitazone, AMG 131, MBX2044, mitoglitazone, lobeglitazone, IDR-105, troglitazone, englitazone, ciglitazone, adaglitazone, darglitazone that enhance insulin action (e.g., by insulin sensitization) including insulin, and insulin mimetics (e.g., insulin degludec, insulin glargine, insulin lispro, insulin detemir, insulin glulisine and inhalable formulations of each), thus promoting glucose utilization in peripheral tissues; 5) glucagon-like-peptides including DPP-IV inhibitors (e.g., alogliptin, omarigliptin, linagliptin, vildagliptin and sitagliptin) and Glucagon-Like Peptide-1 (GLP-1) and GLP-1 agonists and analogs (e.g., exenatide (BYETTA and ITCA 650 (an osmotic pump inserted subcutaneously that delivers an exenatide analog over a 12-month period; Intarcia, Boston, Mass.)) and GLP-1 receptor agonists (e.g., dulaglutide, semaglutide, albiglutide, exenatide, liraglutide, lixisenatide, taspoglutide, CJC-1131, and BIM-51077, including intranasal, transdermal, and once-weekly formulations thereof); and 6) and DPP-IV-resistant analogues (incretin mimetics), PPAR gamma agonists, PPAR alpha agonists such as fenofibric acid derivatives (e.g., gemfibrozil, clofibrate, ciprofibrate, fenofibrate, bezafibrate), dual-acting PPAR agonists (e.g., ZYH2, ZYH1, GFT505, chiglitazar, muraglitazar, aleglitazar, sodelglitazar, and naveglitazar), pan-acting PPAR agonists, PTPIB inhibitors (e.g., ISIS-113715 and TTP814), SGLT inhibitors (e.g., ASP1941, SGLT-3, empagliflozin, dapagliflozin, canagliflozin, BI-10773, PF-04971729, remogloflozin, TS-071, tofogliflozin, ipragliflozin, and LX-4211), insulin secretagogues, angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, valsartan, candesartan, olmesartan, telmesartan); and the like.

### Subjects

Subjects suitable for treatment with a method of the present disclosure include individuals having a metabolic disorder. Subjects suitable for treatment with a method of the present disclosure include obese individuals. Subjects suitable for treatment with a method of the present disclosure include individuals having type 2 II diabetes. Subjects suitable for treatment with a method of the present disclosure include individuals having diabetic retinopathy. Subjects suitable for treatment with a method of the present disclosure include individuals having non-alcoholic fatty liver disease (NAFLD). Subjects suitable for treatment with a method of the present disclosure include individuals having non-alcoholic steatohepatitis (NASH).

In some cases, individuals having and/or diagnosed as having non-alcoholic fatty liver disease (NAFLD) are specifically excluded. In some cases, individuals having and/or diagnosed as having non-alcoholic steatohepatitis (NAS) are specifically excluded.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of treating a metabolic disorder in an individual, the method comprising administering to the individual an effective amount of a compound of Formula I:

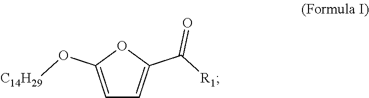

wherein: R¹ is R ¹ is —O—R², —O—R³OR², OR³—OC(O)—N(R⁵)R⁶, —O—R³—N(R⁵)R⁶, —O—R³—N(R⁴)C(O)OR⁵, —O—R³C(O)OR⁵, —O—R³C(O)N(R⁵)R⁶ or- N(R⁵)S(O)₂—R⁴;
each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; each R³ is independently an optionally substituted alkylene chain; R⁴ is optionally substituted alkyl, optionally substituted aryl, optionally substituted aralkyl, optionally substituted heteroaryl or optionally substituted heteroarylalkyl; each R⁵ is independently hydrogen, alkyl, optionally substituted cycloalkyl, optionally substituted aryl or optionally substituted aralkyl; each R⁶ is alkyl, optionally substituted cycloalkyl, optionally substituted aralkyl or —R³C(O)OR⁴; or any R⁵ and R⁶, together with the nitrogen to which they are both attached, form an optionally substituted N-heterocyclyl or an optionally substituted N-heteroaryl; as a single stereoisomer or as a mixture thereof or a pharmaceutically acceptable salt thereof.

Aspect 2. The method of aspect 1, wherein the compound is 5-(tetradecyloxy)-2-furoic acid.

Aspect 3. The method of aspect 1 or aspect 2, wherein the metabolic disorder is insulin resistance, hyperglycemia, type 2 diabetes mellitus, obesity, fatty liver disease, glucose intolerance, hyperinsulinemia, metabolic syndrome, or hypertension.

Aspect 4. The method of any one of aspects 1-3, wherein the metabolic disorder comprises insulin resistance.

Aspect 5. The method of any one of aspects 1-3, wherein the metabolic disorder comprises metabolic syndrome.

Aspect 6. The method of any one of aspects 1-3, wherein the metabolic disorder comprises type 2 diabetes mellitus.

Aspect 7. The method of any one of aspects 1-6, wherein the individual has a body mass index>30.0.

Aspect 8. The method of any one of aspects 1-7, wherein said administering results in a serum insulin level in a normal range.

Aspect 9. The method of any one of aspects 1-7, wherein said administering results in a blood glucose level in a normal range.

Aspect 10. The method of any one of aspects 1-9, further comprising administering at least one additional therapeutic agent.

Aspect 11. The method of aspect 10, wherein the at least one additional therapeutic agent is insulin, an insulin analog, a biguanidine, or a thiazolidinedione.

Aspect 12. The method of any one of aspects 1-11, wherein said administering is via oral administration.

Aspect 13. The method of any one of aspects 1-12, wherein the compound of Formula I is administered daily.

Aspect 14. The method of any one of aspects 1-12, wherein the compound of Formula I is administered once per week.

Aspect 15. The method of any one of aspects 1-12, wherein the compound of Formula I is administered via controlled delivery.

Aspect 16. The method of aspect 15, wherein the compound of Formula I is present in an implantable delivery device.

Aspect 17. A method of treating metabolic syndrome in an individual, the method comprising administering to the individual an effective amount of a compound of Formula I:

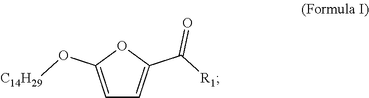

wherein: R¹ is R¹ is —O—R², —O—R³OR², —O—R³OC(O)—N(R⁵)R⁶, —O—R³—N(R⁵)R⁶, —O—R³—N(R⁴)C(O)OR⁵, —O—R³C(O)OR⁵, —O—R³—C(O)N(R⁵)R⁶ or —N(R⁵)S(O)₂—R⁴;
each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; each R³ is independently an optionally substituted alkylene chain; R⁴ is optionally substituted alkyl, optionally substituted aryl, optionally substituted aralkyl, optionally substituted heteroaryl or optionally substituted heteroarylalkyl; each R⁵ is independently hydrogen, alkyl, optionally substituted cycloalkyl, optionally substituted aryl or optionally substituted aralkyl; each R⁶ is alkyl, optionally substituted cycloalkyl, optionally substituted aralkyl or —R³—C(O)OR⁴; or any R⁵ and R⁶, together with the nitrogen to which they are both attached, form an optionally substituted N-heterocyclyl or an optionally substituted N-heteroaryl; as a single stereoisomer or as a mixture thereof or a pharmaceutically acceptable salt thereof.

Aspect 18. The method of aspect 17, wherein the compound is 5-(tetradecyloxy)-2-furoic acid.

Aspect 19. The method of aspect 17 or aspect 18, wherein said administering is via oral administration.

Aspect 20. The method of any one of aspects 17-19, wherein the compound of Formula I is administered daily.

Aspect 21. The method of any one of aspects 17-19, wherein the compound of Formula I is administered once per week.

Aspect 22. The method of any one of aspects 17-19, wherein the compound of Formula I is administered via controlled delivery.

Aspect 23. The method of aspect 22, wherein the compound of Formula I is present in an implantable delivery device.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl. picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Dose Dependent Effect of TOFA in Choline-Deficient High Fat Diet (CDAHFD)-Induced Non-Alcoholic Fatty Liver Discase (NAFLD) Progression in Mice

### Methods

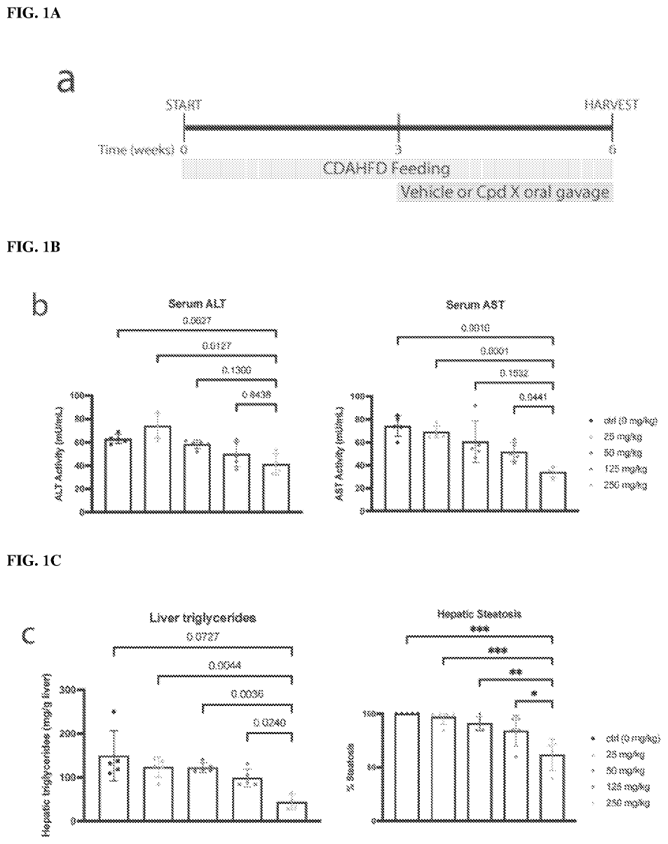

Six-week-old C57BL/6 male mice were fed with a L-Amino Acid Diet With 60 kcal % Fat With 0.1% Methionine and No Added Choline (Research Diets A06071302i) for three weeks before introduction of either vehicle (control) or TOFA via oral gavage for an additional three weeks (FIG. 1a). Mice were gavaged orally twice daily with varying doses of TOFA (0, 25, 50, 125, or 250 mg/kg per day). Upon study termination at six weeks, serum and liver samples were collected. Serum liver damage biomarkers, alanine transaminase (ALT) and aspartate transaminase (AST), were measured by commercially available kits (abcam105134 and abcam 105135, respectively). Hepatic triglycerides were detected from liver samples from a commercially available kit (abcam65336). Liver samples were also scored for degree of hepatic steatosis based on their hematoxylin and eosin (H&E) staining.

### Results

Mice orally treated with TOFA showed a dose dependent decrease in scrum ALT and AST activity levels (FIG. 1B). Similarly, lipid levels in the liver decreased in a dose dependent manner based on TOFA concentration, quantitatively shown by the liver triglyceride contents per gram of liver (FIG. 1C, left). This is supported by the observation of a decreased degree of hepatic steatosis correlating to an increased dosage of TOFA (FIG. 1C, right). These findings indicate that TOFA is well tolerated and specifically decreases lipid accumulation in the liver.

FIG. 1A-1C illustrates the dose dependent effect of TOFA treatment in mice with CDAHFD induced NAFLD progression. FIG. 1A shows the experimental time course. Mice were fed with CDAHFD for 3 weeks before oral treatment with different concentrations of TOFA for 3 weeks. FIG. 1B is a graph illustrating activity levels of liver damage biomarkers, alanine aminotransferase (ALT), on the left, and aspartate aminotransferase (AST), on the right. FIG. 1C is a graph illustrating the hepatic lipid status through a quantitative measure of liver triglycerides, on the left, and a scored assessment of hepatic steatosis from hematoxylin and eosin (H&E)-stained liver section, on the right. Brown-Forsythe and Welch one-way ANOVA statistical tests were used to determine statistical significance.

### Example 2. Rescue Effect of TOFA in 60% kcal Fat Diet Induced Early-Stage Hepatic Steatosis and Associated Metabolic Disorders in Mice

### Methods

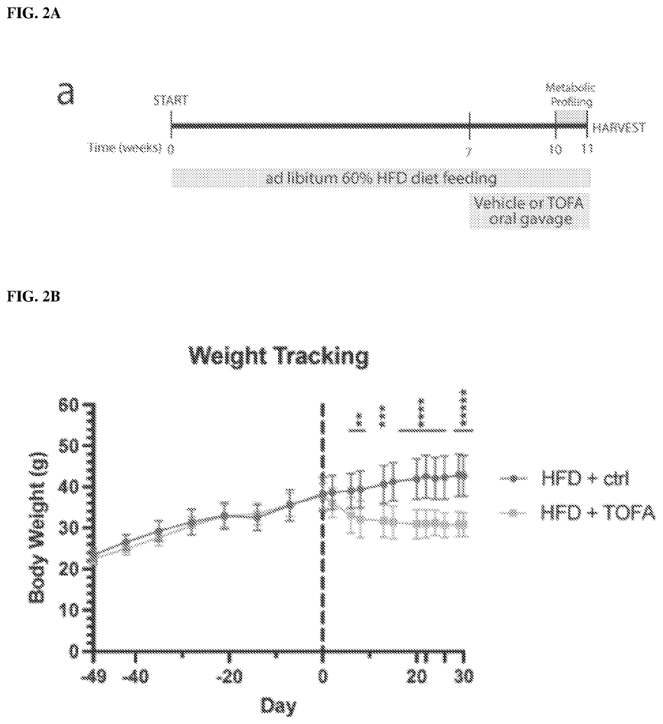

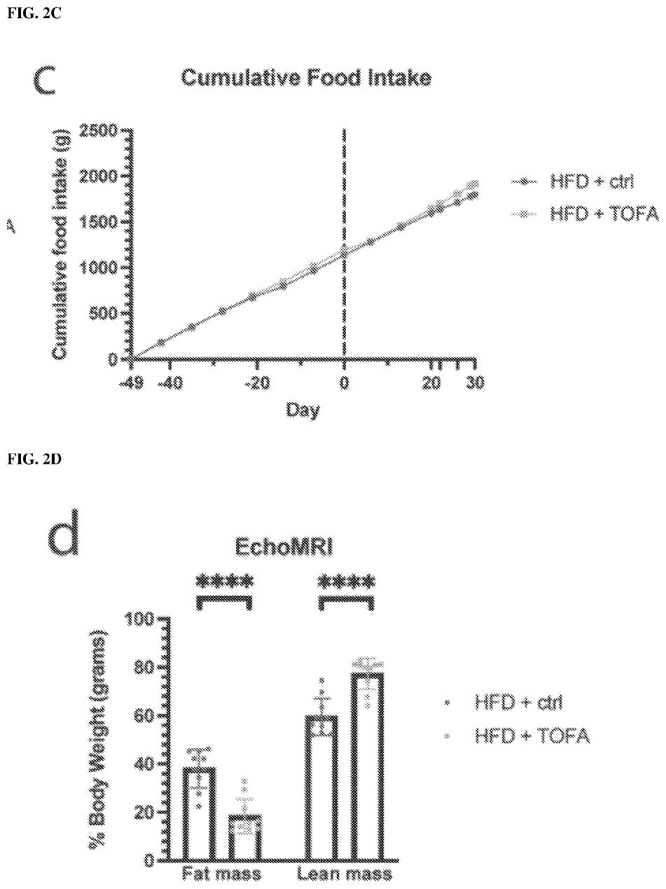

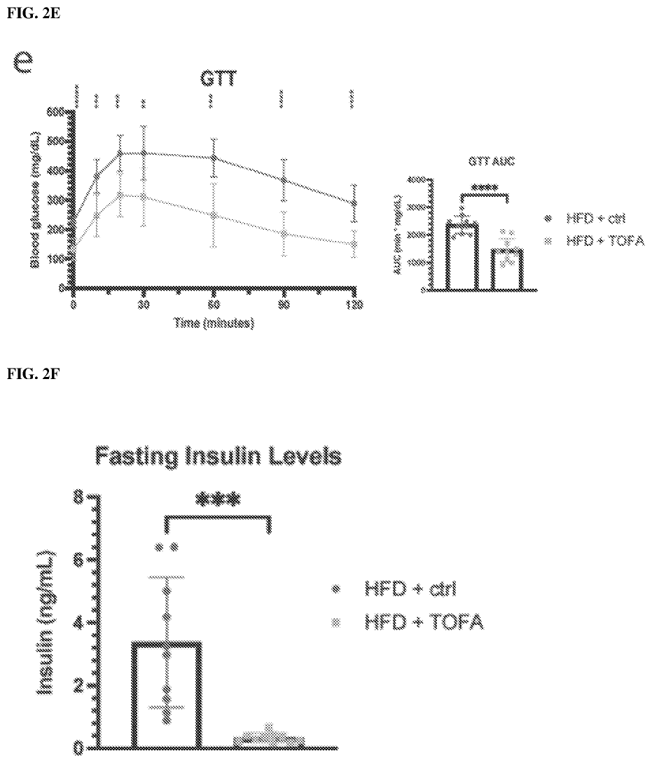

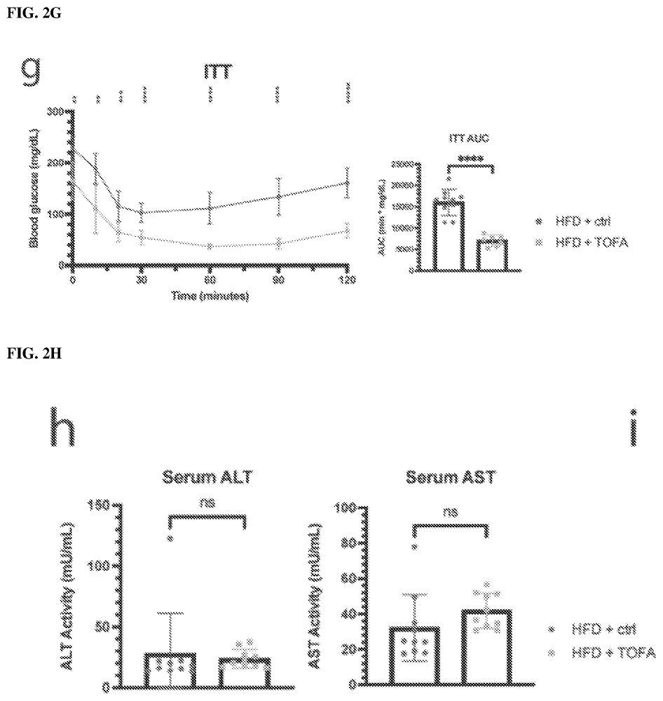

Six-week-old C57BL/6 male mice were fed with a 60 kcal % fat diet (Research Diets A12492i) for seven weeks before introduction of either vehicle (control) or 250 mg/kg initial loading dose of TOFA daily via twice daily oral gavage for one week (FIG. 2A). Mice were then treated with either vehicle (control) or 125 mg/kg maintenance dose of TOFA daily via twice daily gavage for three additional weeks. Mice weights were tracked during the entire time course. After three weeks of treatment, an intraperitoneal glucose tolerance test (IP-GTT) and insulin tolerance test (IP-ITT) were performed. Prior to these tests, mice were fasted for six hours before a challenge of either 1 g/kg D-glucose or 0.75 U/kg HumulinR (Eli Lilly) and blood glucose levels were monitored for two hours (Bayer Contour Next). Venous fasting blood was collected through the tail vein and used to measure fasting insulin levels by commercially available ELISA kits (CrystalChem90080). Prior to harvest, mice underwent whole body composition analysis (EchoMRI™ 2012). Upon study termination at 11 weeks, serum and liver samples were collected. Serum liver damage biomarkers, ALT and AST, were measured by commercially available kits (abcam 105134 and abcam105135, respectively). Hepatic and serum triglyceride levels were measured from a commercially available kit (abcam65336).

### Results

Upon treatment with TOFA, mice exhibited weight loss compared to the vehicle control treated mice, showing the efficacy of TOFA as a weight loss and fat reduction agent, without impacting food intake (FIG. 2B, FIG. 2C). This is supported by the significant reduction of fat mass and increase in lean mass, relative to whole body weight, in TOFA-treated mice (FIG. 2D). The IP-GTT showed TOFA-treated mice were more glucose tolerant via improved glucose clearance overtime (FIG. 2E). Additionally, TOFA-treated mice had vast improvements in fasting blood glucose levels as well as significantly lower fasting insulin levels (FIG. 2E, 2F). The IP-ITT highlights the improved insulin sensitivity via glucose clearance response as a result of TOFA treatment on mice (FIG. 2G). Whilst not being bound by theory, the inhibition of ACC activity by TOFA and subsequent decrease in fatty acid synthesis may contribute to the improvement in these metabolic parameters in glucose homeostasis by reductions in ectopic lipids in the liver that contribute to NAFLD pathology and progression (e.g., insulin resistance). Serum analysis revealed no significant changes in serum ALT and AST biomarkers (FIG. 2H). This could be accounted for the abbreviated time course of dietary-induced damage from the high fat diet. Relative to other diet-induced NAFLD timelines and diets, this dietary time course most closely mimics early-stage metabolic dysregulation (e.g., obesity complications, diabetes mellitus, metabolic syndrome) which is strongly associated with NAFLD progression. However, TOFA treatment significantly reduced both hepatic and serum levels of triglycerides (FIG. 2I). As speculated, the total decrease in lipid circulation in the body may contribute to overall improved metabolic homeostasis. Thus, these results show that TOFA is an effective agent in reducing early stage NAFLD associated metabolic dysregulation by decreasing lipid load in the body.

FIG. 2A-2I illustrate the effect of TOFA treatment on 60% kcal fat diet induced metabolic syndrome and early stage NAFLD progression. FIG. 2A shows the experimental timeline. Mice were fed with a 60% kcal fat diet for seven weeks before oral treatment of TOFA (one week loading dose of 250 mg/kg then three weeks maintenance dose of 125 mg/kg delivered daily over two doses) for 4 weeks. Metabolic profiling began in the final week of treatment. FIG. 2B is a graph tracking the weight changes over the experimental time course. FIG. 2C is a graph tracking cumulative intake of 60% kcal fat diet over the experimental time course. FIG. 2D is a graph illustrating the changes in fat and lean mass, relative to body weight, as measured by EchoMRI. FIG. 2E is a graph illustrating blood glucose levels over time during an intraperitoneal challenge of glucose (IP-GTT), on the left. The area under the curve representation is shown on the right. FIG. 2F is a graph illustrating fasting serum insulin levels. FIG. 2G is a graph illustrating blood glucose levels over time during an intraperitoneal challenge of insulin (IP-ITT), on the left. The area under the curve representation is shown on the right. FIG. 2H is a graph illustrating activity levels of liver damage biomarkers, alanine aminotransferase (ALT), on the left, and aspartate aminotransferase (AST), on the right. FIG. 2I is a graph illustrating body lipid status through a quantitative measure of liver triglycerides, on the left, and a quantitative measure of serum lipid levels, on the right.

### Example 3. Rescue Effect of TOFA in CDAHFD Induced Late-Stage NAFLD/NASH Progression in Mice

### Methods

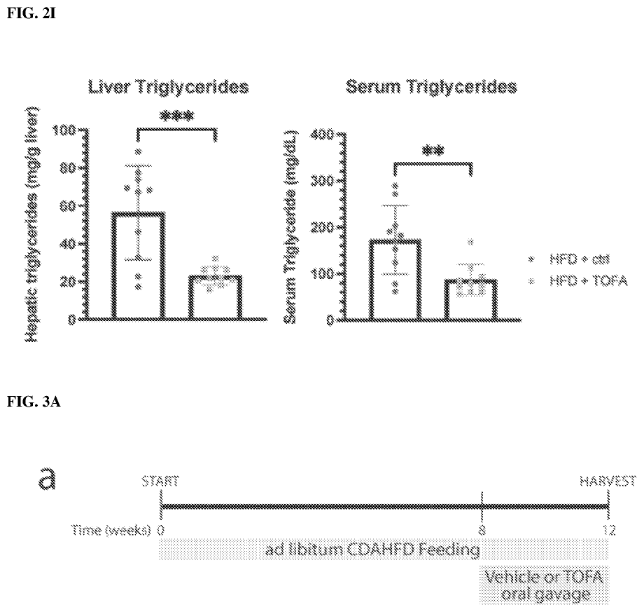

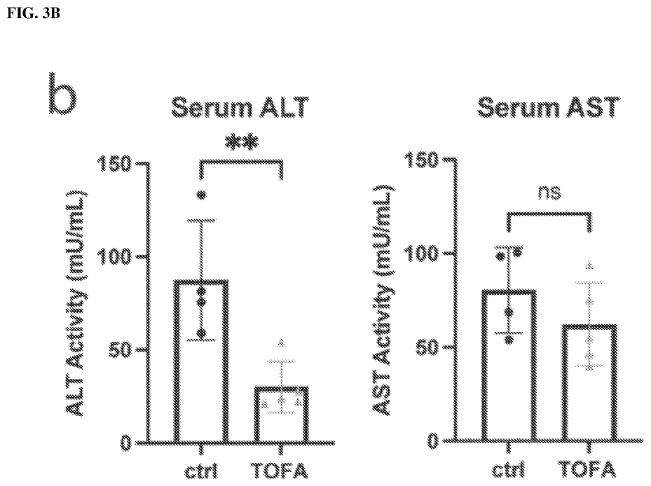

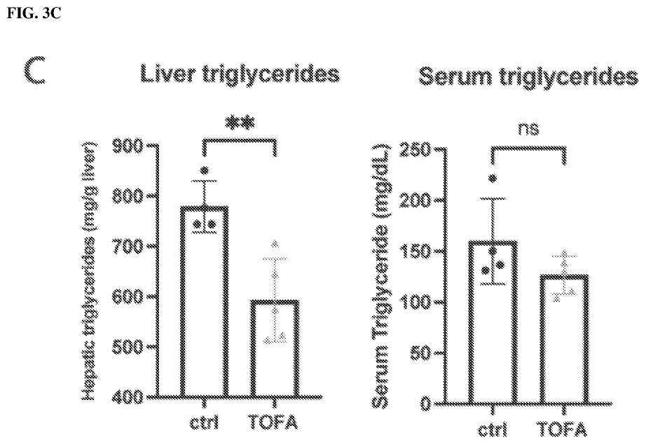

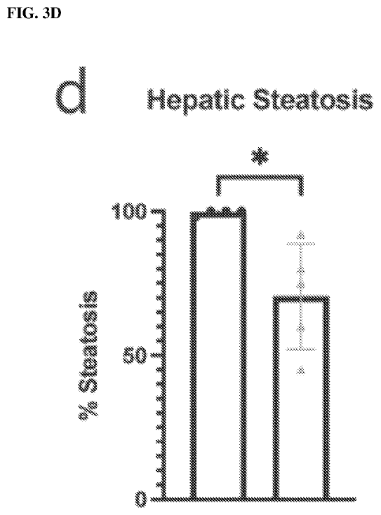

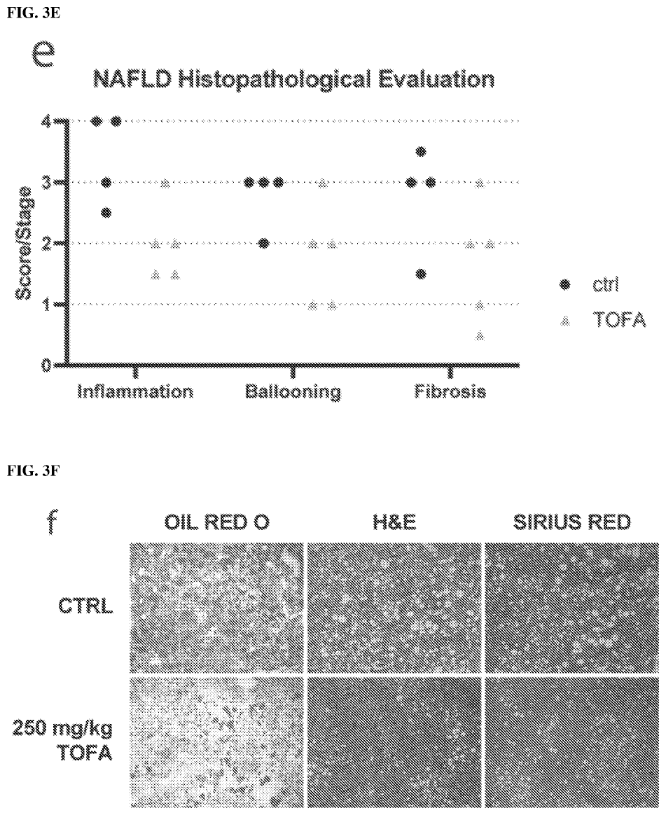

Six-week-old C57BL/6 male mice were fed with a L-Amino Acid Diet With 60 kcal % Fat With 0.1% Methionine and No Added Choline (Research Diets A06071302i) for eight weeks before introduction of either vehicle (control) or 250 mg/kg of TOFA daily via twice daily controlled oral gavage for an additional four weeks (FIG. 3A). Upon study termination at 12 weeks, serum and liver samples were collected. Serum liver damage biomarkers, ALT and AST, were measured by commercially available kits (abcam105134 and abcam 105135, respectively). Hepatic and serum triglyceride levels were measured from a commercially available kit (abcam65336). Liver samples were fixed in 4% paraformaldehyde and embedded in paraffin blocks for sectioning and subsequently stained with hematoxylin and eosin (H&E) or Picrosirius Red with a fast green dye background. Frozen fresh liver tissue was embedded in OCT, sectioned, and subsequently stained with Oil Red O.

### Results

Mice treated with TOFA showed a significant decrease in serum ALT activity levels and an averaged reduction in serum AST levels (FIG. 3B). Additionally, TOFA treatment reduced liver triglyceride content and there was no increase in serum triglyceride levels (FIG. 3c). Histopathological evaluation presented decreased degree of hepatic steatosis in TOFA treated mice as well as reductions in other disease pathological markers such as inflammation, ballooning, and fibrosis as scored by the metric outlined in Kleiner, et al. (2005) (FIG. 2D, FIG. 2E). Histological staining revealed that TOFA treatment presented a reduction in lipid content by Oil Red O, decreased cytoplasmic vacuolations by H&E, and decreased fibrosis by Picrosirius Red staining (FIG. 2F). Overall, these results indicate TOFA is effective in ameliorating late stage NAFLD progression and NASH pathology while minimizing potential adverse metabolic effects previously reported with other ACC inhibitors, such as elevated serum triglyceride levels.

FIG. 3A-3F show the rescue effect of TOFA treatment CDAHFD induced late stage NAFLD/NASH progression in mice. FIG. 3A shows the experimental timeline. Mice were fed with a CDAHFD for 8 weeks before oral treatment of TOFA (250 mg/kg daily over two doses) for four weeks. FIG. 3B is a graph illustrating activity levels of liver damage biomarkers, alanine aminotransferase (ALT), on the left, and aspartate aminotransferase (AST), on the right. FIG. 3C is a graph illustrating body lipid status through a quantitative measure of liver triglycerides, on the left, and a quantitative measure of serum lipid levels, on the right. FIG. 3D is a graph illustrating the scored assessment of hepatic steatosis from hematoxylin and eosin-stained liver sections. FIG. 3E is a graph illustrating the scored histopathological assessment of hepatic inflammation, hepatocyte ballooning, and fibrosis from hematoxylin and eosin and Sirius red stained liver sections. FIG. 3F is a panel showing representative images of Oil Red O, hematoxylin and eosin, and Sirius red stained liver sections. Scale bar is 50 μm. Student's unpaired t-tests were used to determine statistical significance

### Example 4. Whole Genome RNA Sequencing of Liver Samples From CDAHFD-Induced Late Stage NAFLD/NASH Mice Treated With Vehicle or TOFA

### Methods

Liver samples were collected from mice in Example 3 (see above). Total RNA was extracted from liver tissues (Qiagen RNeasy kit), cDNA libraries were constructed (Roche KAPA HyperPrep kit), sequenced on the NovaSeq6000 platform (Novogene), and a downstream analysis pipeline was performed. Differentially expressed genes (DEGs) were defined by at least one log2-fold change (2-fold in linear scale) with an FDR cutoff of 0.05. For validation, total RNA was used to prepare cDNA (BioRAD iScript™ Reverse Transcription) according to manufacturer's protocol and quantitative polymerase chain reaction (qPCR) was performed with fast SYBR Green Mix (ThermoFisher) on the QuantStudio6 System (Applied Biosystems). Protein expression levels were measured by western blot using antibodies against very low density lipoprotein receptor (VLDLR) (AF2258, 1:2000) and btubulin (CST2146, 1:1000). Membranes were developed using a horse radish peroxidase (HRP) secondary antibody (1:5000) and visualized using an enhanced chemiluminescent HRP substrate (Thermo34577).

### Results

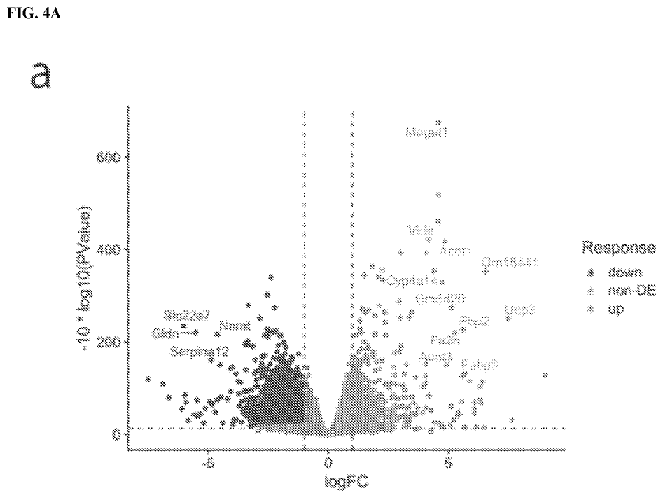

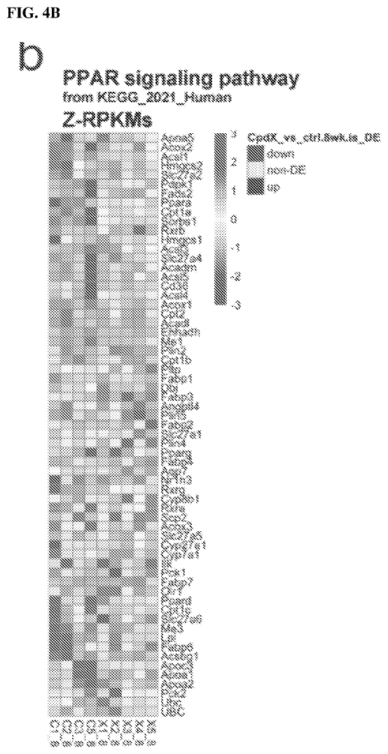

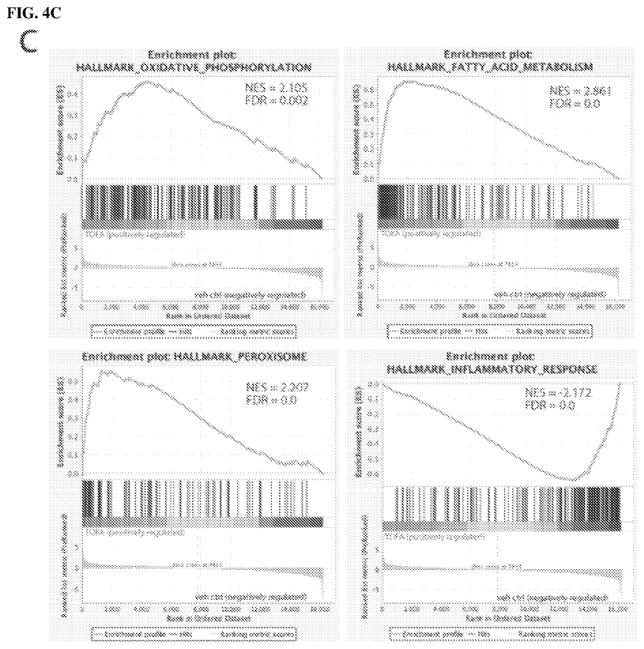

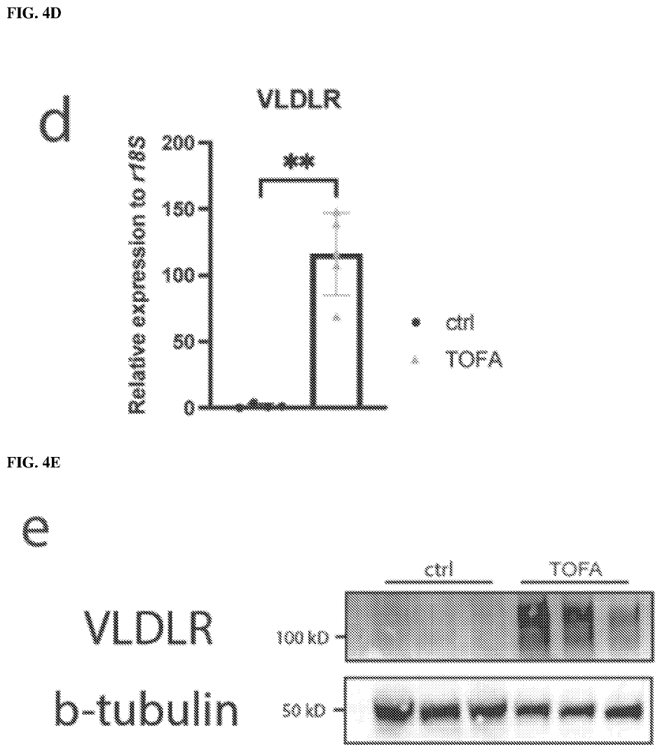

Intriguingly, RNA-seq analysis of liver samples revealed a potential role of TOFA in affecting the transcriptional activity of the peroxisome proliferator-activated receptors (PPARs) nuclear hormone receptor superfamily, specifically PPAR-alpha. Several of the highest upregulated genes seen with TOFA treatment are canonical PPAR-alpha target genes (FIG. 4A). Specific analysis of the PPAR signaling pathway (from the 2021 KEGG human database) showed that TOFA treatment upregulated various genes under PPAR transcriptional control (FIG. 4B). This observation was further confirmed by various GSEA heat maps of significantly upregulated genes in processes such as oxidative phosphorylation, fatty acid metabolism, and peroxisomes, all of which are processes tightly associated with the action of the PPAR signaling pathway (FIG. 4C). There is also an observed negative correlation with the inflammatory response from TOFA treatment, supporting the safety and efficacy of TOFA treatment to reduce inflammatory markers of NAFLD pathology (FIG. 4C, bottom right). Amongst the upregulated genes from TOFA treatment, there was a significant increase in expression of the very low density lipoprotein receptor (VLDLR) gene, a PPAR-alpha target gene (FIG. 4D). This increase in mRNA expression levels is translated to an increase in protein expression levels of VLDLR in TOFA treated liver samples (FIG. 4E). Previous studies have demonstrated the role of VLDLR upregulation via PPAR-alpha agonism (e.g., fenofibrate) to mediate triglyceride lowering effects. This preliminary data bridges the potential relationship between TOFA action of VLDLR upregulation and PPAR-alpha agonist action from the fenofibrate-PPAR-alpha-VLDLR signaling axis to induce the triglyceride lowering effect. Thus, without being bound by theory, the use of TOFA or derivatives in treating fatty liver disease and its associated metabolic dysregulation may utilize a multifaceted polypharmacology approach through ACC inhibition and PPAR agonism.

FIG. 4A-4E show the results of RNA sequencing of liver samples from mice treated with TOFA from CDAHFD-induced late stage NAFLD/NASH. FIG. 4A is a volcano plot of differentially upregulated and downregulated genes. Differential genes at an FDR cutoff of 0.05 and a 2-fold change cutoff. FIG. 4B is a heat map illustrating the upregulated and downregulated genes in the PPAR signaling pathway, taken from the KEGG-2021-human database. Columns represent samples and rows represent genes. Colors indicate gene expression level (log2 RPKM) relative to average expression across all samples. FIG. 4C is a GSEA plot of differentially regulated genes enriched in several hallmark gene sets: oxidative phosphorylation (top left), fatty acid metabolism (top right), peroxisome (bottom left), and inflammatory response (bottom right). Normalized enrichment score (NES) and false discovery rate (FDR) are shown for each hallmark. FIG. 4D is a graph illustrating the expression of mouse liver VLDLR mRNA relative to expression of r18S mRNA. FIG. 4E is a western blot image of VLDLR and beta-tubulin protein expression levels in mouse liver samples. Student's unpaired t-tests were used to determine statistical significance.

### Example 5. Tolerability and Toxicity of TOFA in Mice

### Methods

Eleven-week-old C57BL/6J male mice were fed with PicoLab Rodent Diet (Purina 5053), or chow diet, ad libitum while being treated with either control or 125 mg/kg, BID by oral gavage for three weeks. Mice weights were tracked during the entire time course. Upon study termination, serum and liver samples were collected. Serum biomarkers such as blood urea nitrogen (BUN) and creatinine were assessed by commercially available kits (abcam83362 and Cayman700460, respectively). Additionally, hepatic and serum triglyceride and cholesterol levels were measured by commercially available kits (abcam65336 and abcam65390, respectively).

### Results

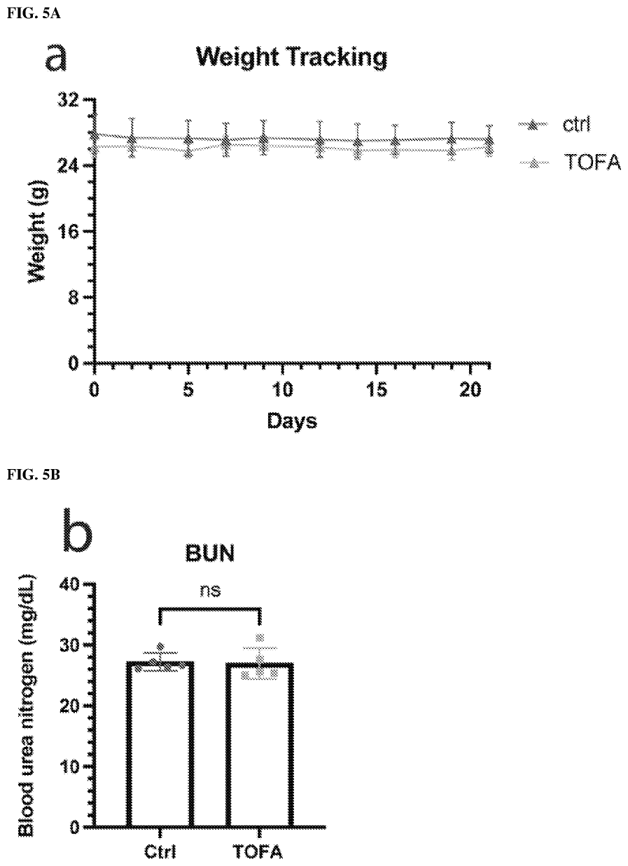

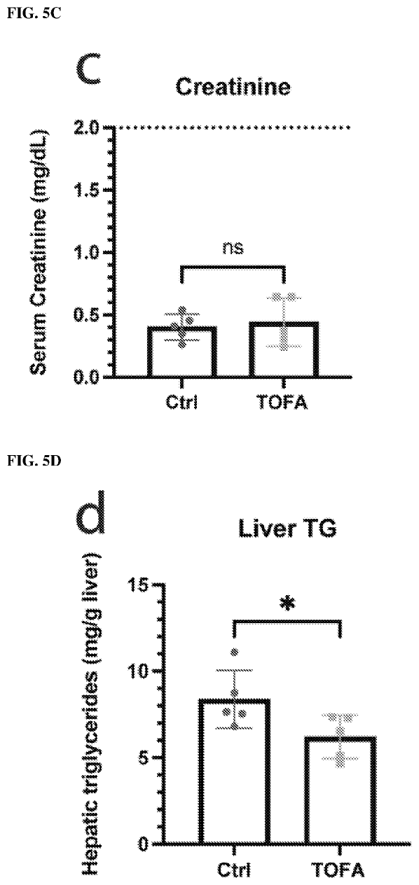

Over the course of treatment, there was no observed weight change in mice treated with TOFA and no weight difference between TOFA-treated and vehicle-treated mice (FIG. 5A). Analysis of scrum biomarkers such as BUN (FIG. 5B) and creatinine (FIG. 5C) indicated there is no drug-induced kidney damage. There is an observed decrease in liver triglyceride levels (FIG. 5D), which is characteristic for this class of metabolic modulator. Furthermore, there is no significant change in serum triglyceride levels (FIG. 5E), which is a unique observation amongst other small molecules targeting the same enzymes (ACC1/2). No changes in cholesterol levels in the liver (FIG. 5F) or in serum (FIG. 5G) were observed. These findings indicate that TOFA is orally bioavailable, suitable for BID or less frequent dosing regimens, and well tolerated in mice.

FIG. 5A-5B illustrates the safety and tolerability profile of TOFA treatment in ad libitum fed chow mice. FIG. 5A is a graph tracking the weight changes over the experimental time course. FIG. 5B is a graph illustrating blood urea nitrogen (BUN) levels in serum. FIG. 5C, is a graph illustrating creatinine levels in serum. FIG. 5D is a graph illustrating a quantitative measure of liver triglycerides. FIG SE is a graph illustrating a quantitative measure of serum triglycerides. FIG. 5F is a graph illustrating a quantitative measure of liver cholesterol. FIG. 5G is a graph illustrating a quantitative measure of total serum cholesterol. Student's unpaired t-tests were used to determine statistical significance.

### Example 6. Dose-dependent Effect of TOFA in 60% HFD Mouse Model of Diet-Induced Obesity (DIO)

### Methods

Six-week-old C57BL/6J male mice were fed with a 60 kcal % fat diet (Research Diets A12492i) for ten weeks before introduction of either vehicle (control) or TOFA, at varying doses, for an additional two weeks. TOFA was orally administered in doses of either 12.5, 25, 62.5, or 125 mg/kg, BID. Mice weights and food intake were monitored during the treatment time course. Prior to harvest, mice underwent whole body composition analysis (EchoMRI™ 2012). Upon study termination at 12 weeks, serum and liver samples were collected. Hepatic and serum triglyceride and cholesterol levels were measured by commercially available kits (abcam65336 and abcam65390, respectively). Additional liver samples were fixed in 4% paraformaldehyde and embedded in paraffin blocks for sectioning and subsequently stained with hematoxylin and eosin (H&E).

### Results

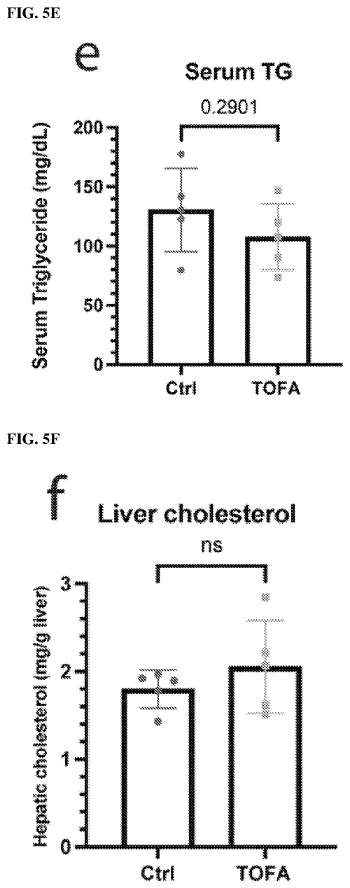

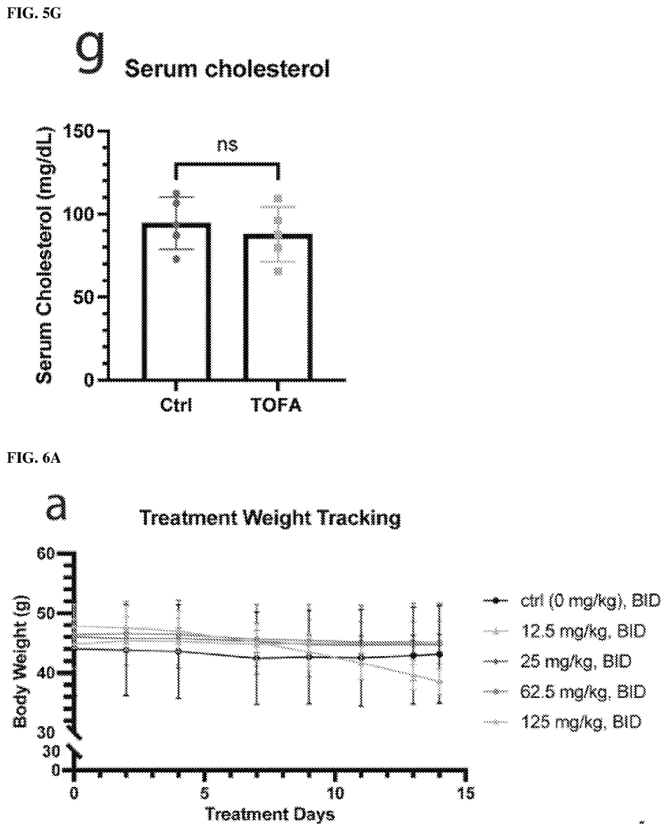

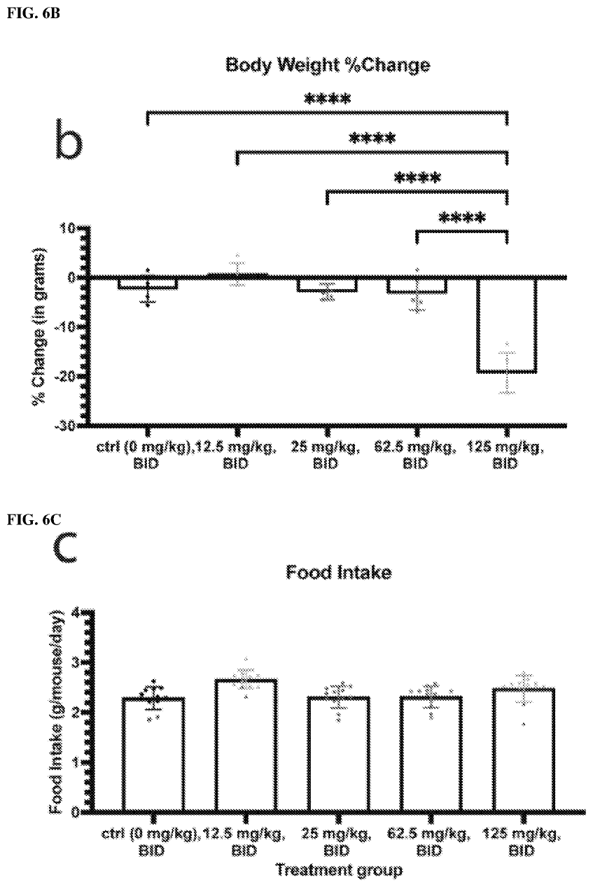

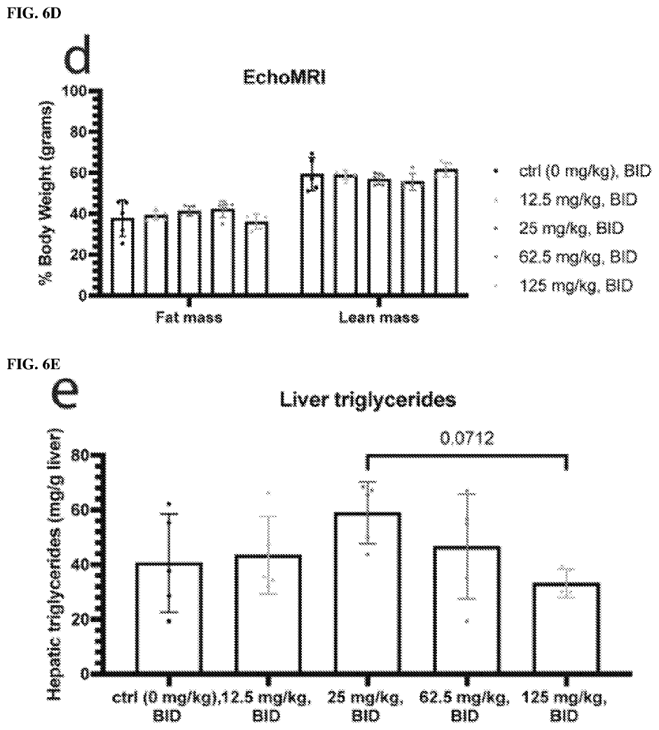

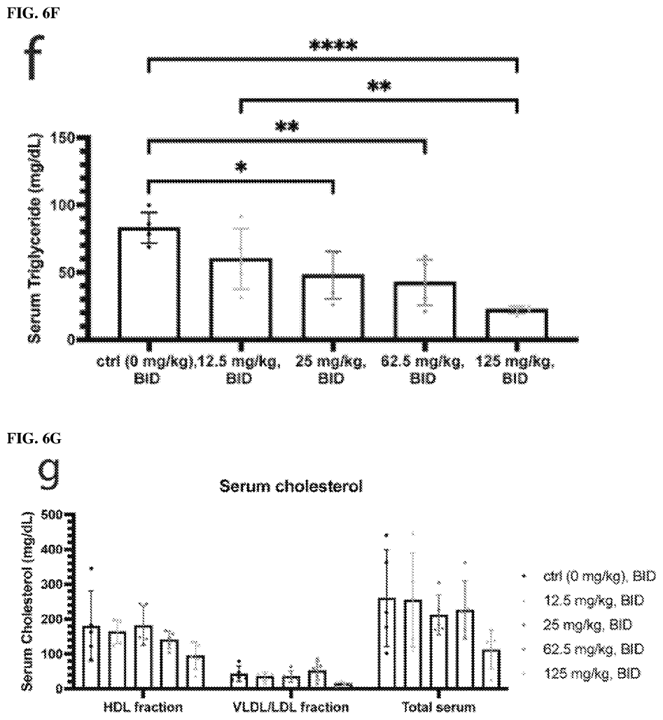

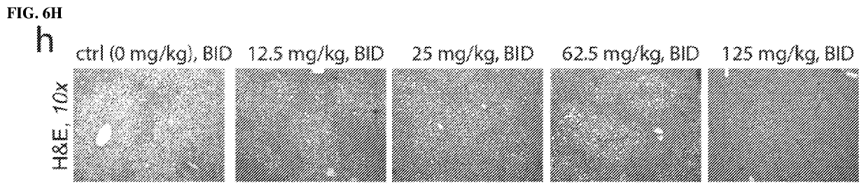

Upon treatment with TOFA, mice exhibited weight loss at the highest dose (125 mg/kg, BID), demonstrating an efficacious dose (FIG. 6A). Dosing at lower concentrations resulted in smaller or negligible changes in body weight, comparable to vehicle control-treated mice (FIG. 6B). Changes in body weight between treatment group was not attributed to food intake changes during treatment, which remained constant throughout the time course (FIG. 6C). Body composition analysis revealed a decrease in fat mass as a percentage of body weight in the highest dose treatment group with no changes in overall lean mass between all treatment groups, attributing weight changes to decreases in fat mass (FIG. 6D). Analysis of liver triglycerides further support the notion of changes in lipid levels in the body, with striking decreases in liver triglyceride levels in the highest dosing treatment regimens (FIG. 6E). Most strikingly, there is a dose-dependent decrease in serum triglyceride levels (FIG. 6F) and serum cholesterol levels, specifically for both the HDL and VLDL/LDL fractions (FIG. 6G). Histological analysis of liver sections revealed an improvement in hepatic steatosis in a dose-dependent manner and provides further support of 125 mg/kg, BID, as the most effective dosing regimen (FIG. 6H). Overall, these results show TOFA behaving in a dose-dependent manner to address multiple features of metabolic syndrome and further support the metabolic benefits conferred upon TOFA treatment in a mouse diet-induced model of metabolic syndrome (see Example 2).

FIG. 6A-6H illustrate the dose dependent effect of TOFA treatment in mice with diet-induced obesity with a 60% HFD dietary model. FIG. 6A is a graph tracking weight changes over the treatment time course. FIG. 6B is a graph illustrating total percentage body weight change from the beginning to the end of the treatment time course. FIG. 6C is a graph illustrating food intake of 60% kcal fat diet over the experimental time course. FIG. 6D is a graph illustrating the changes in fat and lean mass, relative to body weight, as measured by EchoMRI. FIG. 6E is a graph illustrating a quantitative measure of liver triglycerides. FIG. 6F is a graph illustrating a quantitative measure of serum triglycerides. FIG. 6G is a graph illustrating a quantitative measure of HDL cholesterol. VLDL/LDL cholesterol, and total serum cholesterol. FIG. 6H is a panel showing representative images of hematoxylin and eosin staining of liver sections at 10× magnification. Brown-Forsythe and Welch one-way ANOVA statistical tests were used to determine statistical significance.

### Example 7. Transcriptional Analysis of Liver Samples From 60% HFD Mouse Model of Diet Induced Obesity and Associated Metabolic Disorders

### Methods

Six-week old C57BL/6J male mice were fed with a 60 kcal % fat diet (Research Diets A12492i) for seven weeks before introduction of either control or 125 mg/kg, BID, initial loading dose of TOFA daily via controlled oral gavage for one week (FIG. 2A). Mice were then treated with either vehicle (control) or 62.5 mg/kg, BID, maintenance dose of TOFA daily via oral gavage for three additional weeks. Upon study termination at 11 weeks, liver samples were collected. Total RNA was extracted from liver tissues (Qiagen RNeasy kit 74106), cDNA libraries were constructed (Roche KAPA HyperPrep kit), sequenced on the NovaSeq6000 platform (Novogene), and a downstream analysis pipeline was performed. Differentially expressed genes (DEGs) were defined by at least one log2-fold change (2-fold in linear scale) with an FDR cutoff of 0.05. For validation, total RNA was used to prepare CDNA (BioRAD iScript™ Reverse Transcription) according to manufacturer's protocol and qPCR was performed with fast SYBR Green Mix (ThermoFisher A25742) on the QuantStudio6 System (Applied Biosystems).

### Results

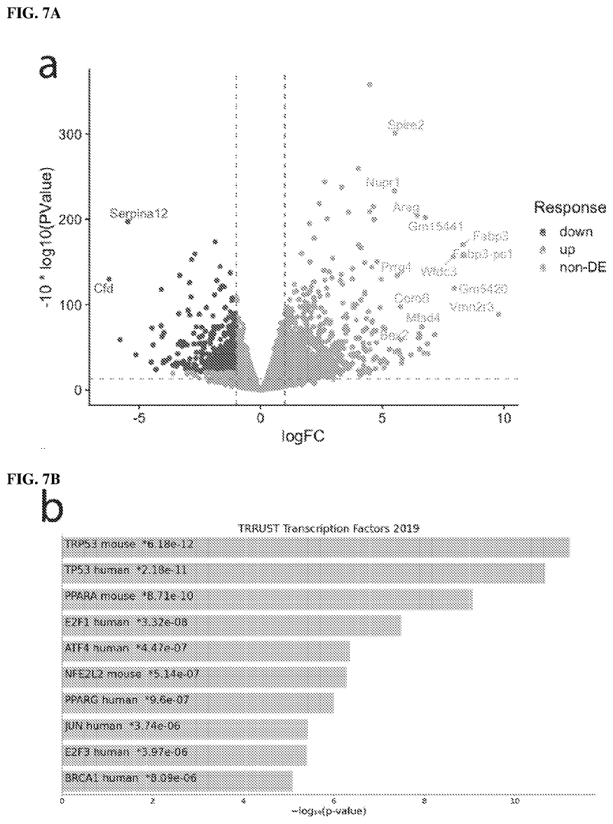

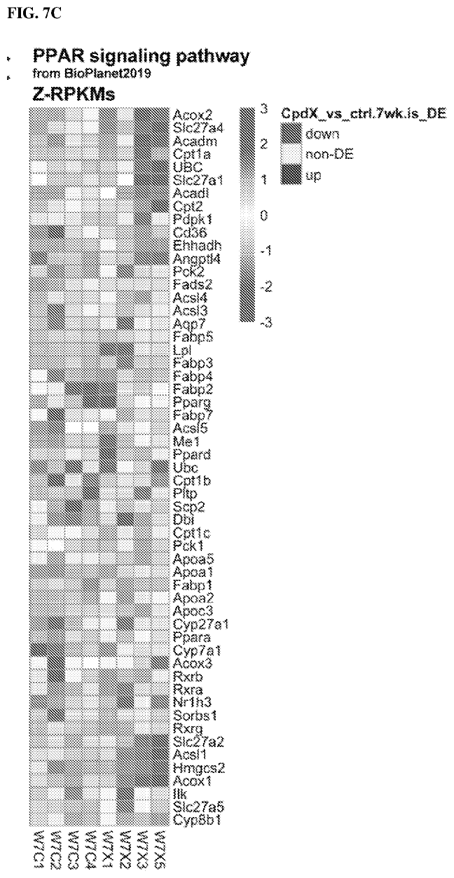

In support of results from Example 4, RNAseq analysis of liver samples revealed a potential role of TOFA in affecting the transcriptional activity of the peroxisome proliferator-activated receptor (PPAR) signaling network, an observation conserved in a different diet-induced model of severe metabolic disease. Several of the highest upregulated genes seen with TOFA treatment are canonical PPAR-alpha target genes, as well as notable downregulated genes that are negatively regulated by PPAR-alpha activation (FIG. 7A). Transcriptional regulatory relationship analysis of upregulated genes from the TRRUST Transcription Factors 2019 reference database reveals that PPARA is one of the major acting factors in transcriptional regulation after TOFA treatment (FIG. 7B). This observation was further supported by a specific analysis of genes in the PPAR signaling pathway from the BioPlanet 2019 database highlighting that TOFA treatment upregulated various genes under PPAR transcriptional control (FIG. 7C). Various genes from RNAseq analysis identified as downstream PPAR targets were confirmed by reverse transcription-qPCR (RT-qPCR) to be upregulated from TOFA treatment and involved in various processes in lipid homeostasis, such as fatty acid beta-oxidation, acyl-CoA processing, and lipoprotein uptake and metabolism (FIG. 7D). These results support a similar transcriptional activity signature of TOFA regardless of diet-challenge mouse models and stage of metabolic disease severity.

FIG. 7A-7D show a transcriptional analysis of genes changed in the liver from TOFA treatment in a 60% HFD dietary-induced obesity mouse model. FIG. 7A is a volcano plot of differentially upregulated and downregulated genes. Differential genes at an FDR cutoff of 0.05 and a 2-fold change cutoff. FIG. 7B is a bar table showing p-values of some of the most upregulated transcription factors signature from the pool of upregulated genes from the TRRUST Transcription Factor 2019 reference database. FIG. 7C is a heat map illustrating the upregulated and downregulated genes in the PPAR signaling pathway, taken from the BioPlanet 2019 database. Columns represent samples and rows represent genes. Colors indicate gene expression level (log2 RPKM) relative to average expression across all samples. FIG. 7D is a graph illustrating the expression of mouse liver mRNA levels of selected genes with PPAR transcriptional regulation, grouped by functional commonalities, relative to expression of r18s mRNA. Student's unpaired t-tests were used to determine statistical significance.

### Example 8. In-depth Investigation of Rescue Effect of TOFA in CDAHFD Induced Late Stage NAFLD/NASH Progression in Mice

### Methods

Six-week-old C57BL/6J male mice were fed with a L-amino acid diet with 60 kcal % fat with 0.1% methionine and no added choline (CDAHFD; Research Diets A06071302i) for eight weeks. Ten subjects were randomly assigned to either a vehicle (control) treatment or a 125 mg/kg. BID, of TOFA treatment delivered by oral gavage for an additional four weeks. Mice weights were monitored during the entire treatment time course. Upon study termination at 12 weeks, serum and liver samples were collected. Serum liver damage biomarkers, ALT and AST, were measured by commercially available kits (abcam 105134 and abcam 105135, respectively). Hepatic and serum triglyceride levels were measured from a commercially available kit (abcam65336). Total RNA was extracted from liver tissues (Qiagen RNeasy kit 74106), and cDNA was prepared (BioRAD iScript™ Reverse Transcription) according to manufacturer's protocols. Quantitative PCR (qPCR) was performed with fast SYBR Green Mix (ThermoFisher A25742) on the QuantStudio6 System (Applied Biosciences).

### Results

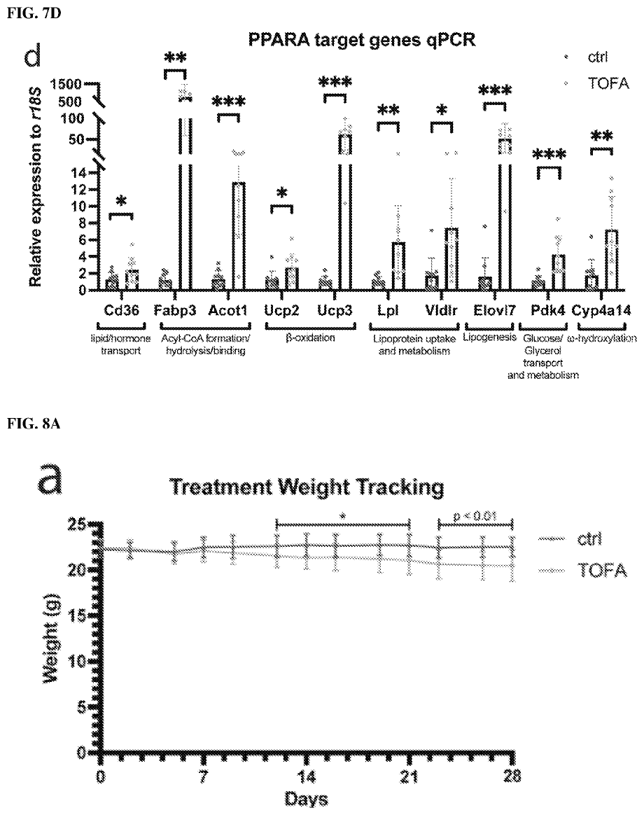

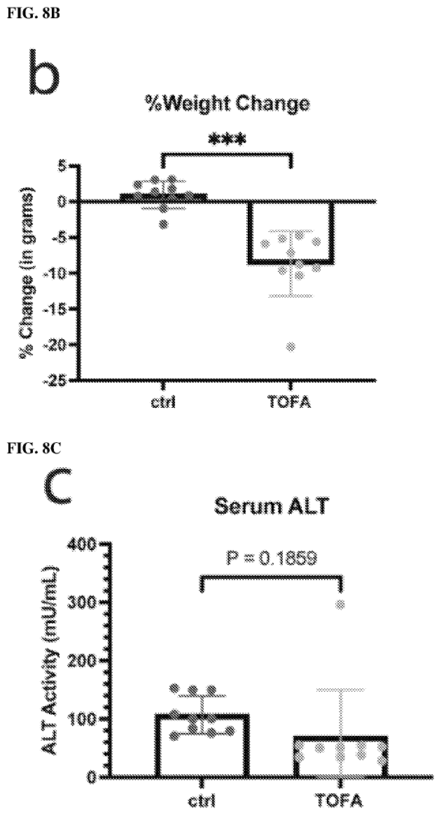

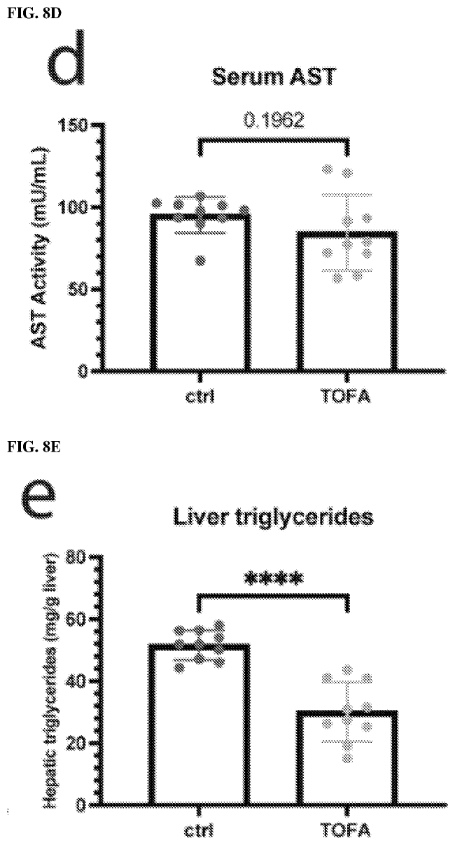

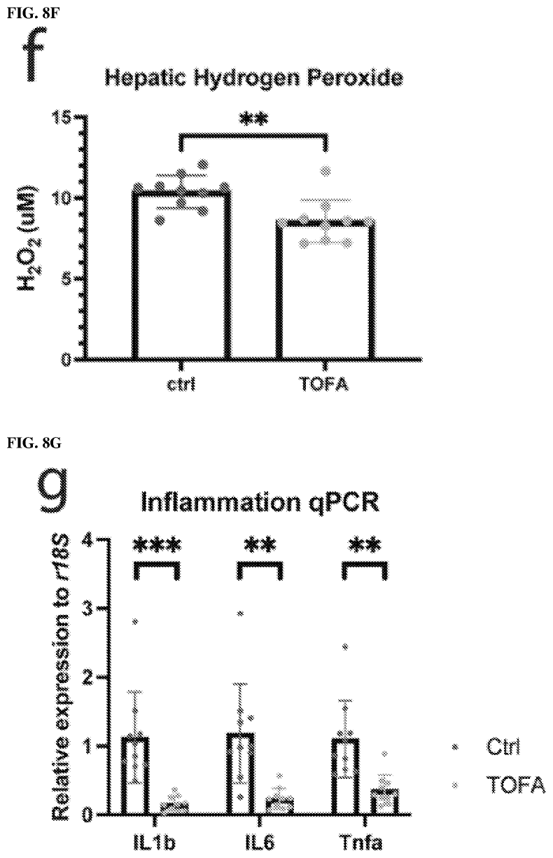

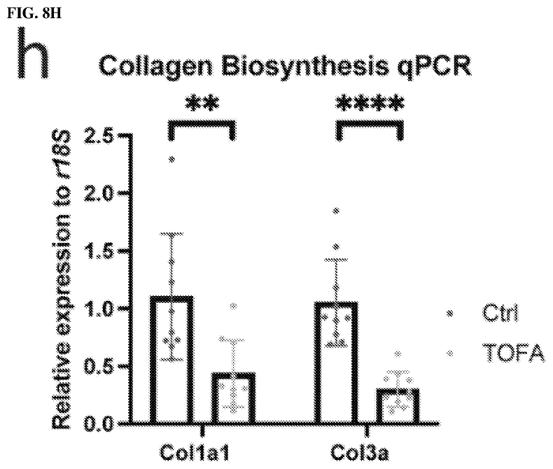

Significant differences in body weight was observed beginning at about ten days after treatment start and the difference grew more significant until study termination (FIG. 8A). TOFA-treated mice had a significant decrease in body weight compared to vehicle control-treated mice (FIG. 8B). Analysis of serum biomarkers for liver damage showed trending decreases in ALT (FIG. 8D) and AST (FIG. 8D). Furthermore, there was a significant decrease in liver triglyceride levels (FIG. 8D) as well as a decrease in hepatic hydrogen peroxide levels in TOFA-treated mice, indicative of a decrease in reactive oxygen species (ROS) (FIG. 8F). Analysis of liver analytes support the notion of improved liver health from TOFA treatment in a diet-induced model of NAFLD/NASH. Inflammation genes, such as Il1b and Tnfa, were assessed by RT-qPCR as biomarkers for inflammation. Results show a decrease in expression of these inflammation-related genes (FIG. 8G). Furthermore, expression of genes involved in collagen biosynthesis was decreased, as assessed by qPCR, indicative of a decrease in fibrosis (FIG. 8H). As seen in Example 4 and Example 7, there is transcriptional activity from the PPAR signaling network that could contribute to TOFA's therapeutic effect. RT-qPCR analysis of select genes with PPAR regulatory elements revealed several genes involved in various processes such as beta-oxidation and lipogenesis upregulated with TOFA treatment (FIG. 8I). These results are all in support and provide further evidence of previous observations (Example 3 and Example 4) of the role of TOFA in addressing multiple aspects of NAFLD/NASH, such as metabolic dysregulation, inflammation, and fibrosis.

FIG. 8A-8I show an in-depth analysis of TOFA treatment efficacy in a CDAHFD dietary-induced mouse model of NAFLD/NASH. FIG. 8A is a graph tracking weight changes over the treatment time course. FIG. 8B is a graph illustrating total percentage body weight change between the start and end of the treatment time period. FIG. 8C is a graph illustrating activity levels of serum alanine aminotransferase (ALT). FIG. 8D is a graph illustrating activity levels of serum aspartate aminotransferase (AST). FIG. 8E is a graph illustrating a quantitative measure of liver triglycerides. FIG. 8F is graph illustrating a quantitative measure of hepatic hydrogen peroxide. FIG. 8G is a graph illustrating the expression levels of mouse liver mRNA related to inflammation relative to expression of r18s mRNA.

### Example 9. TOFA Is Comparable in Efficacy in a Benchmark Study Against Leading Clinical Candidates for NASH Using a Mouse Model of CDAHFD-Induced Late Stage NAFLD/NASH

### Methods

Six-week-old C57BL/6J male mice were fed with a L-amino acid diet with 60 kcal % fat with 0.1% methionine and no added choline (CDAHFD; Research Diets A06071302i) for eight weeks. Mice were randomly assigned to treatment groups of either a vehicle (control), 250 mg/kg of TOFA, 5 mg/kg of Firsocostat (Gilead Sciences), 50 mg/kg of Fenofibrate, or a combined dose of 5 mg/kg Firsocostat and 50 mg/kg Fenofibrate. All treatments were delivered QD by oral gavage for an additional four weeks. Mice weights and food intake were monitored during the treatment time course. Upon study termination at 12 weeks, liver and serum samples were collected. Serum liver damage biomarkers, ALT and AST, were measured by commercially available kits (abcam 105134 and abcam 105135, respectively). Hepatic and serum triglyceride levels were measured from a commercially available kit (abcam65336). Total RNA was extracted from liver tissues (Qiagen RNeasy kit 74106), and cDNA was prepared (BioRAD iScript™ Reverse Transcription) according to manufacturer's protocols. Quantitative PCR (qPCR) was performed with fast SYBR Green Mix (ThermoFisher A25742) on the QuantStudio6 System (Applied Biosciences). Protein expression levels of liver samples were measured by western blot using antibodies against VLDLR (AF2258, 1:2000), CPT1A (CST12252, 1:1000) and Histone H3 (CST9715, 1:1000). Membranes were developed using a HRP secondary antibody (1:5000) and visualized using an enhanced chemiluminescent HRP substrate (Thermo34577). Additional liver samples were fixed in 4% paraformaldehyde and embedded in paraffin blocks for sectioning and subsequently stained with hematoxylin and eosin (H&E).

### Results

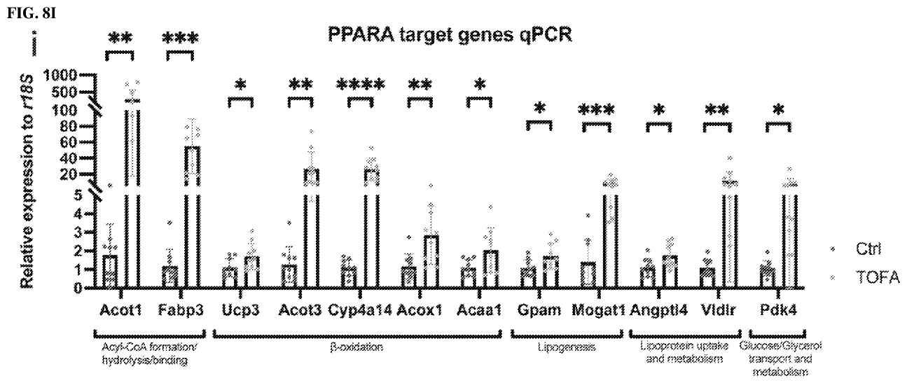

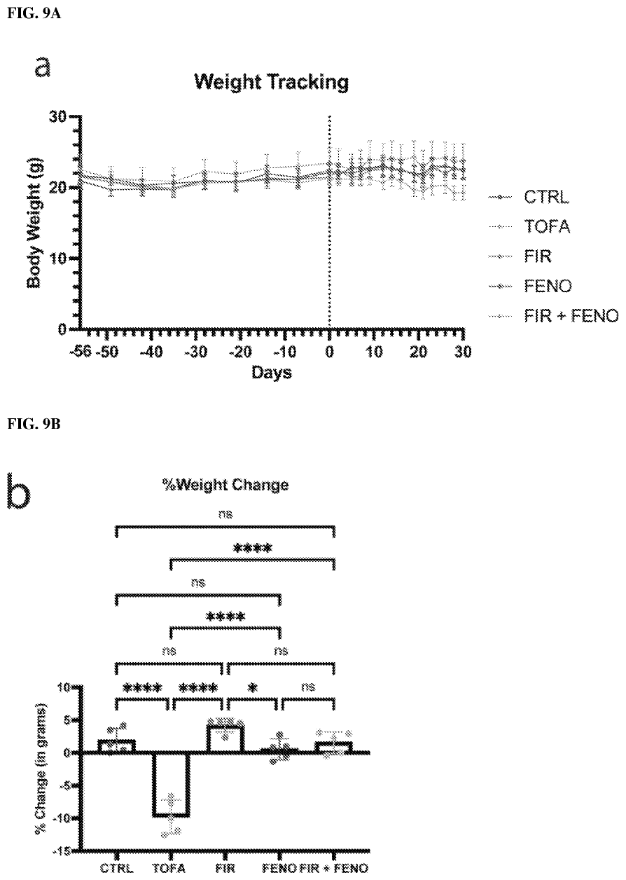

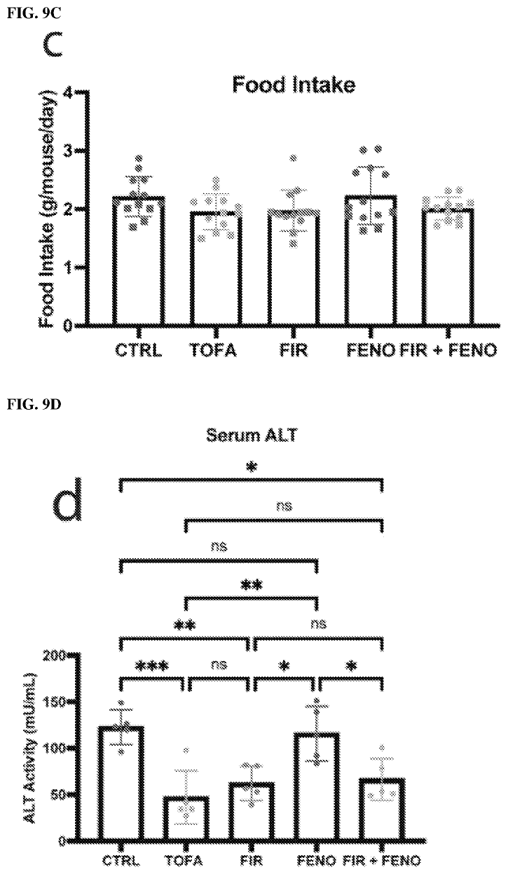

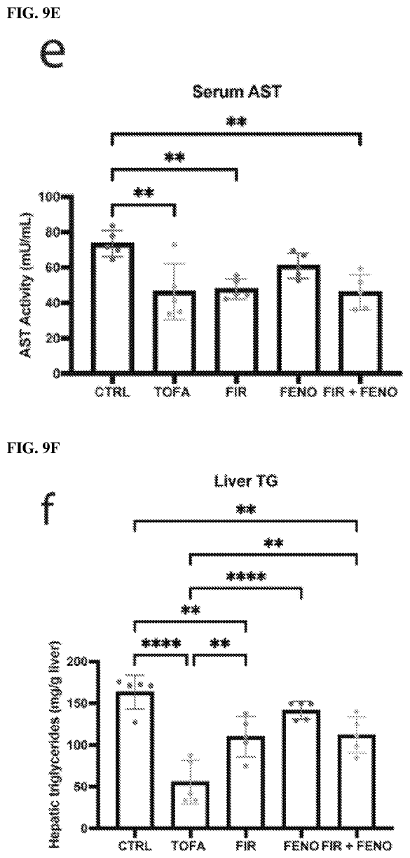

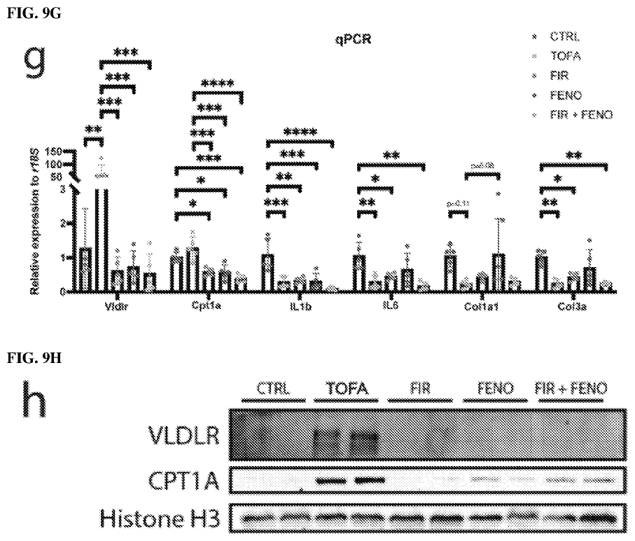

To assess TOFA effect compared with other clinical candidates in the same molecular targeting class, diet-induced NAFLD/NASH mice were treated with optimal dosing of either vehicle control, TOFA, Firsocostat (ACC1/2 inhibitor, Gilead Sciences), Fenofibrate (PPAR-alpha agonist), or a combination of Firsocostat and Fenofibrate. Amongst all treatment regimens, TOFA-treated mice demonstrated significant weight loss whereas all other treatment regimens had comparable body weight changes compared to vehicle control-treated mice (FIG. 9A, FIG. 9B). Changes in body weight were not attributed to changes in food intake (FIG. 9C). Serum analysis of liver damage biomarkers, such as ALT (FIG. 9D) and AST (FIG. 9E), revealed that TOFA treatment resulted in similar levels of ALT and AST decreases as Firsocostat single and combination treatment. Most strikingly, TOFA treatment had a strong decrease in liver triglyceride levels relative to Firsocostat treatments and vehicle control treatment (FIG. 9F). TOFA treatment largely decreased liver damage relative to vehicle control treatment at comparable, or stronger, degrees than Firsocostat, Fenofibrate, or the combination of the two agents. RT-qPCR analysis further showed a specific, comparable response of TOFA treatment relative to the other treatment regimens, such as genes involved in inflammation (e.g., Il1b and Il6) and collagen synthesis (e.g., Col1a1 and Col3a) (FIG. 9G). Furthermore, TOFA treatment induced a unique increase in PPARA downstream targets, such as Vldlr and Cpt1a, which is not observed with the other treatment regimens (FIG. 9G). Western blot analysis of protein expression levels supports the RT-qPCR observations as TOFA treatment increased protein expression levels of VLDLR and CPT1A to higher degrees than the other treatment regimens (FIG. 9H). Histological analysis by H&E staining further supported our observations of decreased levels of steatosis in TOFA-treated groups relative to control and comparable levels of improvement compared to other treatment regimens (FIG. 9I). Taken together, these results are evidence for the use of TOFA in an effective, single regimen dosing that has comparable efficacy against leading clinical candidates in a CDAHFD mouse model of diet-induced NAFLD/NASH.

### Example 10. TOFA Demonstrates Efficacy as a Combination Agent With Other Metabolic Modulating Therapeutics, Such as GLP1-Receptor Agonists

### Methods

Six-week-old C57BL/6J male mice were fed with a L-amino acid diet with 60 kcal % fat with 0.1% methionine and no added choline (CDAHFD; Research Diets A06071302i) for eight weeks. Mice were randomly assigned to treatment groups of either a vehicle (control), 250 mg/kg of TOFA, 5 nmol/kg of Semaglutide (Novo Nordisk), or a combined dose of 250 mg/kg TOFA plus 5 nmol/kg Semaglutide. TOFA was administered by oral gavage. Semaglutide dosing was up-titrated at beginning of treatment regimen and was delivered via subcutaneous injection at the back shoulder/neck flank. All treatments were delivered QD for an additional four weeks. Mice weights and food intake were monitored during the treatment time course. Upon study termination at 12 weeks, liver and serum samples were collected. Serum liver damage biomarkers, ALT and AST, were measured by commercially available kits (abcam105134 and Cayman 701640, respectively). Hepatic and serum triglyceride levels were measured using a commercially available kit (abcam65336). Total RNA was extracted from liver tissues (Qiagen RNeasy kit 74106), and cDNA was prepared (BioRAD iScript™ Reverse Transcription) according to manufacturer's protocols. Quantitative PCR (qPCR) was performed with fast SYBR Green Mix (ThermoFisher A25742) on the QuantStudio6 System (Applied Biosciences). Additional liver samples were fixed in 4% paraformaldehyde and embedded in paraffin blocks for sectioning and subsequently stained with hematoxylin and eosin (H&E) or Sirius Red with a fast green dye background.

Results

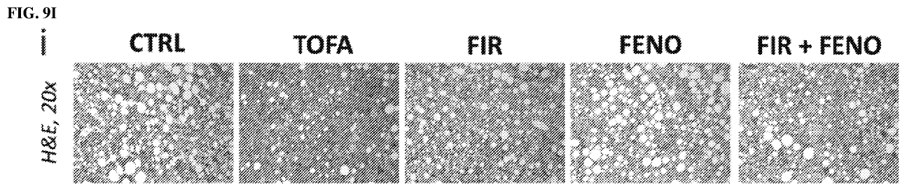

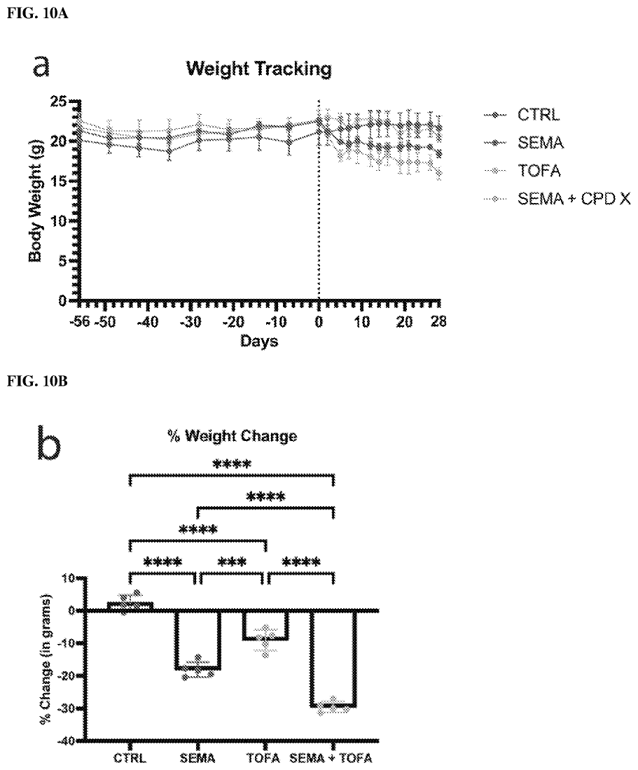

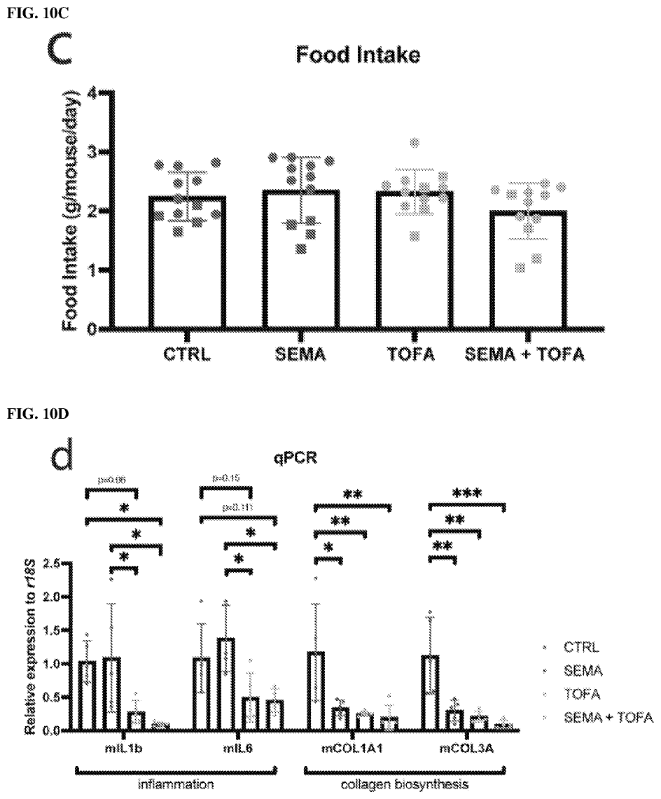

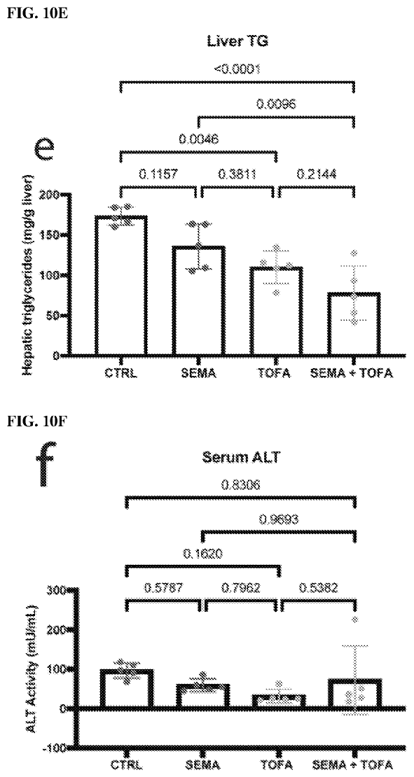

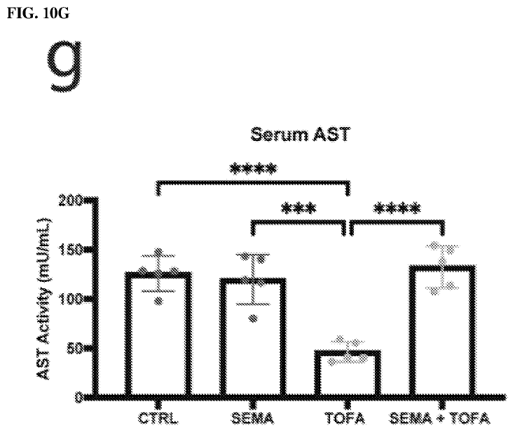

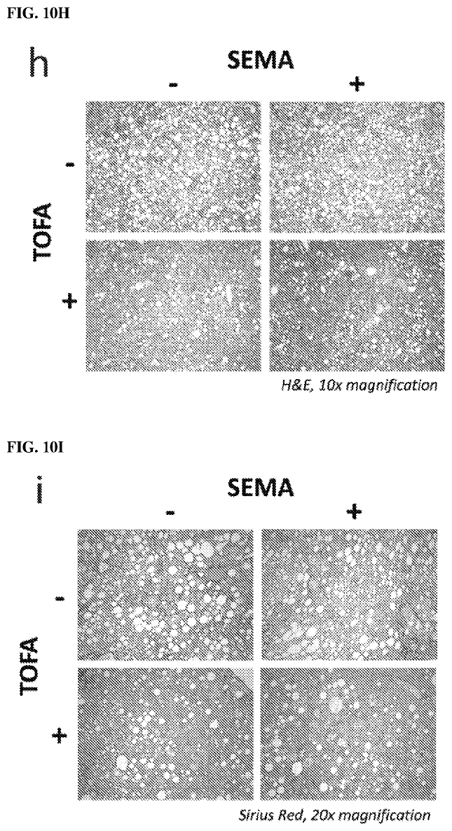

Due to TOFA exhibiting a unique mechanism of action compared to other metabolic modulators, such as GLP-1 receptor agonists, TOFA was used as a combination agent with Semaglutide (Novo Nordisk) to test for efficacy in a CDAHFD mouse model of diet-induced NAFLD/NASH. Upon treatment start, single-treated and combination-treated groups with Semaglutide immediately experienced weight loss, consistent with previous observation of Semaglutide as a weight loss modulator (FIG. 10A). At the end of the treatment time course, it was revealed that TOFA-treated mice experienced a smaller degree of weight loss compared to Semaglutide treated mice, but the combination-treated (TOFA and Semaglutide) mice experienced greater weight loss relative to single agent treatment groups and vehicle control-treated groups (FIG. 10B). Body weight changes were not due to changes in food intake during the treatment time course (FIG. 10C). RT-qPCR analysis of liver samples revealed a specific response of TOFA in addressing inflammation, which was not observed in single Semaglutide-treated mice (FIG. 10D). Analysis of genes involved in collagen biosynthesis by RT-qPCR show strong repressive effectives of both TOFA and Semaglutide in reducing mRNA levels, whereas the combination treatment showed trending further decreases in mRNA expression levels (FIG. 10D). Liver triglyceride analysis revealed that, relative to vehicle control treatment, Semaglutide-treated mice had trending decrease in liver triglyceride levels whilst TOFA treated mice had a stronger, significant reduction in liver triglycerides (FIG. 10E). The combination treatment had the greatest reduction in overall hepatic triglyceride levels (FIG. 10E). Analysis of serum ALT showed similar trends of moderate reductions in ALT levels from single agent treatment regimens and combination treatments (FIG. 10F). Single agent TOFA treatment was the only group to have demonstrated reduction in serum AST levels, which can be attributed to the non-hepatic specific expression of AST that could contribute to serum AST levels, such as kidney and cardiac and skeletal muscle (FIG. 10G). Serum biomarker analysis further support the use of TOFA as an effective therapeutic in decreasing hepatic damage as read out from liver damage biomarkers. Histological analysis by H&E show similar trends as observed in which, relative to control, single Semaglutide treatment presented moderate reductions in steatosis as compared with a greater reduction in steatosis in single TOFA-treated liver sections and the greatest reduction in steatosis (FIG. 10H). Similarly, the aforementioned histological trends are noted in Sirius red staining for fibrotic content (FIG. 101. Altogether. TOFA represents a potential candidate as a combination agent with clinical leads with orthogonal targets and mechanisms. The Semaglutide-TOFA combination results highlight the tolerability of TOFA for combination with no observed drug-drug antagonist effects and combination improvements in addressing shortcomings of single Semaglutide treatment efficacy, such as metabolic and inflammatory aspects, based on preliminary observations.

### Example 11. TOFA Has Unexpected Action Compared to Other Small Molecules in the Same Class, Such as Complementary and Broad Functionalities, Including Activation of PPARA

### Methods

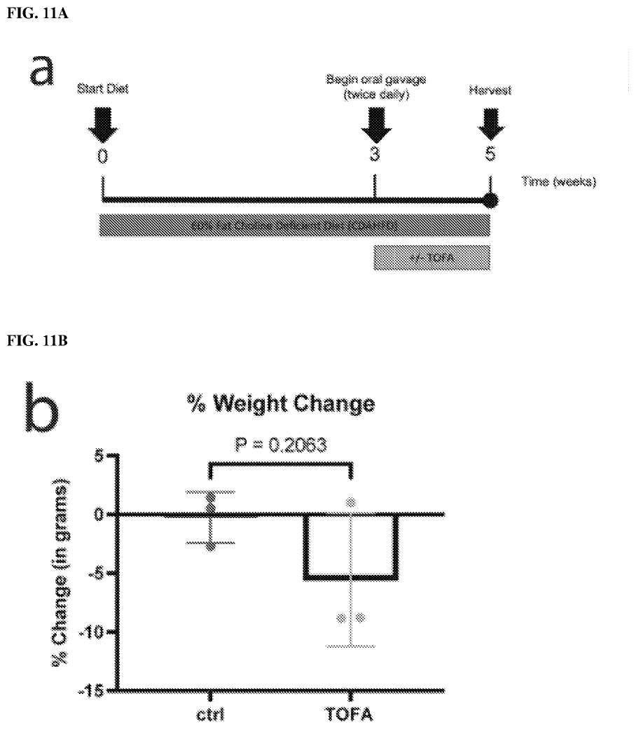

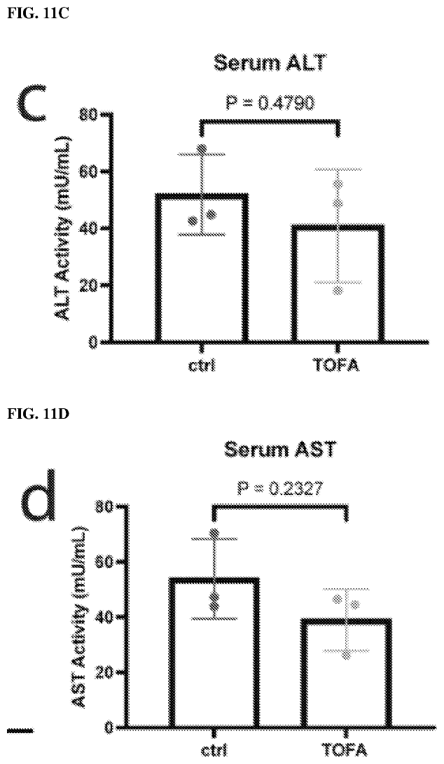

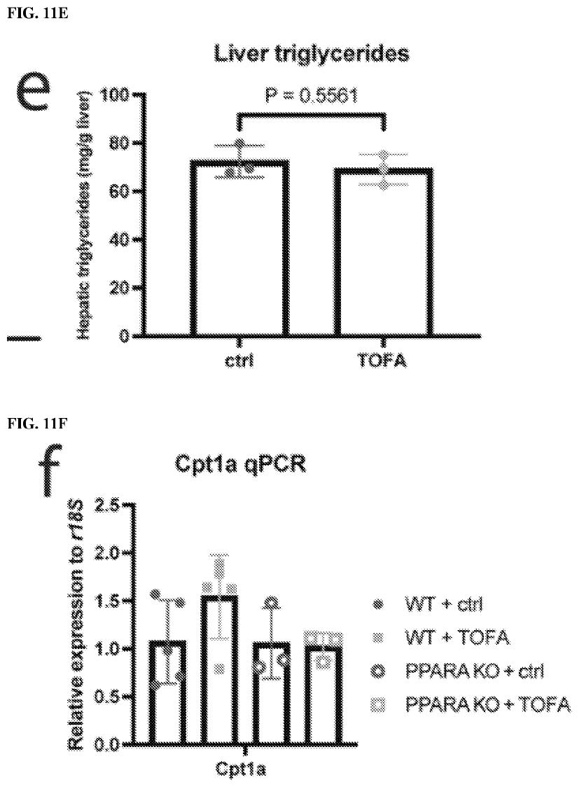

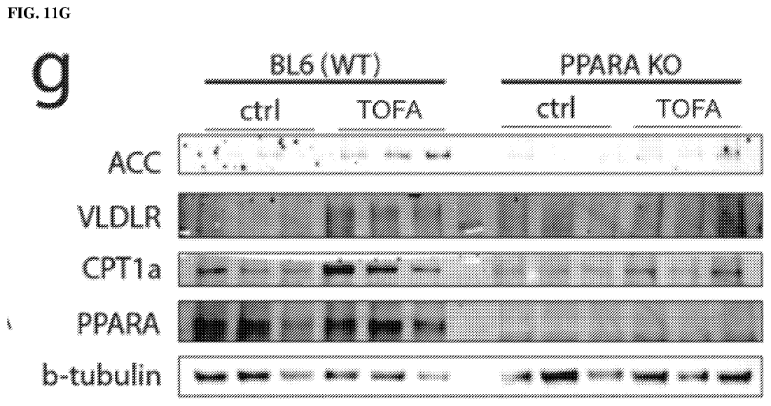

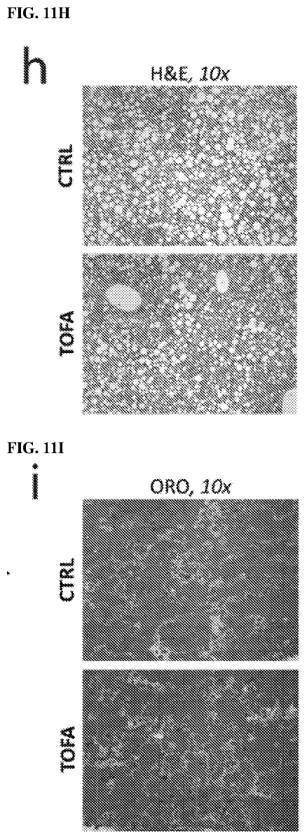

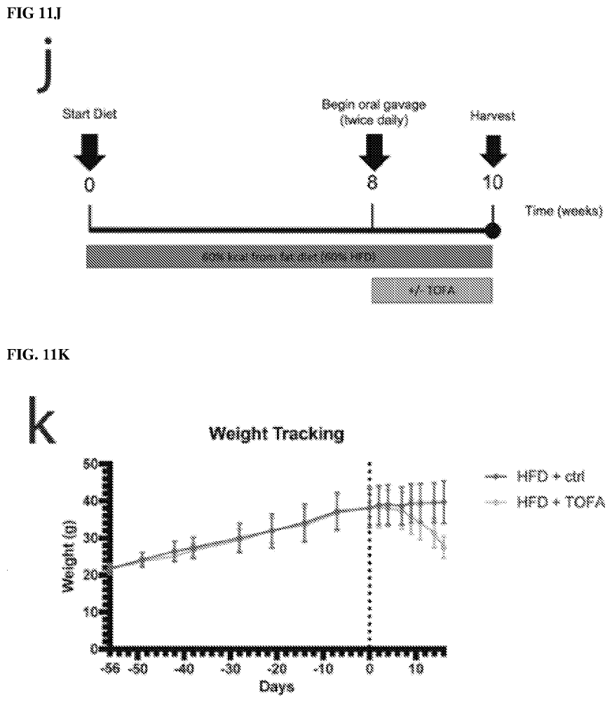

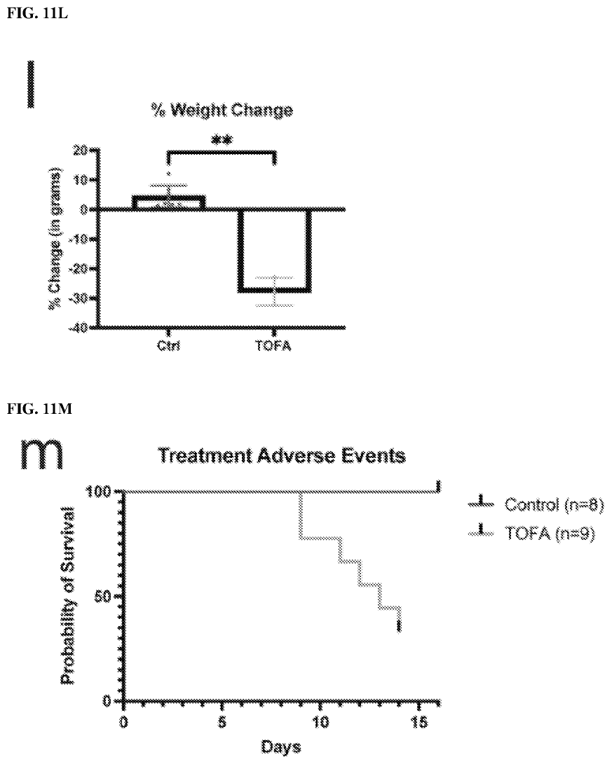

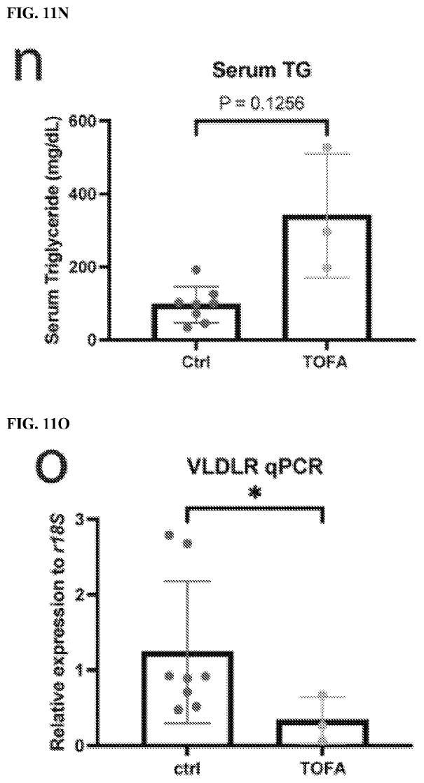

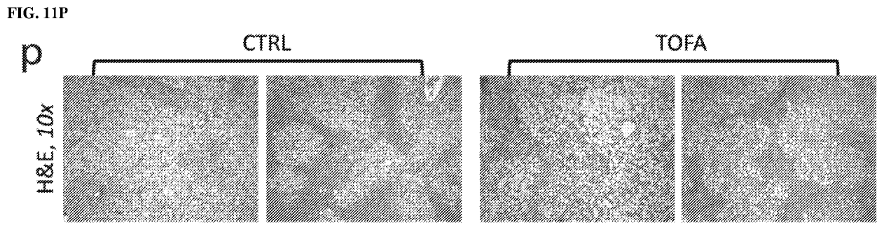

Six-week-old genetic PPARA knockout (PPARA KO) male mice were fed with a L-amino acid diet with 60 kcal % fat with 0.1% methionine and no added choline (CDAHFD; Research Diets A06071302i) for three weeks or fed with a 60 kcal % fat diet (DIO; Research Diets A12492i) for eight weeks before introduction of either vehicle (control) or 125 mg/kg of TOFA, BID, via oral gavage for an additional two weeks (FIG. 11A and FIG. 11J, respectively). C57BL/6J (WT) mice were fed with the CDAHFD and underwent similar experimental conditions and time course. Mice weights and adverse events were monitored during the treatment time course. Upon study termination, serum and liver samples were collected. Serum liver damage biomarkers, ALT and AST, were measured by commercially available kits (abcam105134 and abcam 105135, respectively). Hepatic and serum triglyceride levels were measured from a commercially available kit (abcam65336). Total RNA was extracted from liver tissues (Qiagen RNeasy kit 74106), and cDNA was prepared (BioRAD iScript™M Reverse Transcription) according to manufacturer's protocols. Quantitative PCR (qPCR) was performed with fast SYBR Green Mix (ThermoFisher A25742) on the QuantStudio6 System (Applied Biosciences). Protein expression levels of liver samples were measured by western blot using antibodies against ACC1/2 (CST3662, 1:1000), VLDLR (AF2258, 1:2000), CPT1A (CST12252, 1:1000), PPARA (abcam126285, 1:1000) and beta-tubulin (CST2146, 1:1000). Membranes were developed using a HRP secondary antibody (1:5000) and visualized using an enhanced chemiluminescent HRP substrate (Thermo34577). Additional liver samples were fixed in 4% paraformaldehyde and embedded in paraffin blocks for sectioning and subsequently stained with hematoxylin and eosin (H&E). Frozen fresh liver tissue was embedded in OCT, sectioned, and subsequently stained with Oil Red O.

### Results

In a mouse CDAHFD diet-induced model of NAFLD/NASH with PPARA KO, the overall TOFA treatment regimen caused a trending decrease in weight (FIG. 11B). Analysis of serum biomarkers for liver damage, such as ALT and AST, revealed marginal decreases in ALT and AST levels (FIG. 11E and FIG. 11D, respectively). Furthermore, liver triglyceride analysis showed no notable decrease in hepatic lipid levels (FIG. 11E), by contrast with observations in previous studies in wild-type mice (FIG. 3C and FIG. 8E). RT-qPCR analysis of Cpt1a, a gene involved in fatty acid beta-oxidation, revealed that induction of Cpt1a mRNA was specific to TOFA treatment in wild-type mice and this upregulation response by TOFA treatment was blunted in PPARA KO mice (FIG. 11F). This observation was further supported by Western blot analysis of protein expression levels of select proteins such as CPT1A, VLDLR, and ACC1, all of which are direct transcriptional targets of PPAR-alpha. Protein expression levels of the aforementioned proteins were increased in TOFA treated wild-type mice, but this response was not observed in the TOFA-treated PPARA KO mice relative to each of their vehicle control-treated counterparts (FIG. 11G). Histological analysis by H&E revealed minimal decreases in hepatic steatosis (FIG. 11H) and minimal changes in lipid content by Oil Red O (FIG. 11I). Both of these histological observations are a notable distinct from previous observations with TOFA treatment in wild-type mice (FIG. 3F). These results together provide evidence of the unique mechanistic reliance of TOFA on the PPARA signaling network to mediate its beneficial effects in a diet-induced NAFLD/NASH model. In a diet-induced obesity (DIO) model utilizing a 60% HFD, TOFA treatment in PPARA KO mice caused a decrease in weight upon treatment start (FIG. 11K) and resulted in a significant difference in total body weight change through the treatment course (FIG. 11L). Adverse events were observed in PPARA KO mice treated with TOFA over the two-week treatment period (FIG. 11M). Serum triglyceride analysis of control and TOFA-treated PPARA KO mice revealed elevated serum lipid levels in TOFA-treated mice (FIG. 11N). These observations indicate the mechanistic reliance of TOFA on PPARA to counteract such adverse events given the known role of PPARA for lowering serum triglyceride levels. RT-qPCR analysis of Vldlr, a gene involved in lipoprotein uptake and under PPARA transcriptional control, is shown to have a blunted response in TOFA-treated PPARA KO mice (FIG. 11O), which is unexpected given the consistent upregulation of Vldlr in TOFA-treated HFD-fed wild-type mice (FIG. 7i). Histological comparison between H&E-stained liver sections of control- treated and TOFA-treated PPARA KO mice show minimal changes in hepatic steatosis, further supporting the notion of blunted effects of TOFA treatment in the absence of PPARA (FIG. 11P). To our knowledge, our results show the first empirical evidence of the unexpected mechanistic reliance of TOFA on the PPARA signaling in addressing severe metabolic disease in mouse diet-induced disease models.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a metabolic disorder in an individual, the method comprising administering to the individual an effective amount of a compound of Formula I: 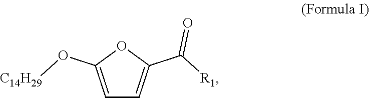
wherein: R¹ is R¹ is —O—R², OR³OR², OR³—OC(O)—N(R⁵)R⁶, —O—R³—N(R⁵)R⁶, —O—R³—N(R⁴)C(O)OR⁵, —O—R³—C(O)OR⁵, —O—R³C(O)N(R⁵)R⁶ or —N(R⁵)S(O)₂—R⁴;
each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; each R³ is independently an optionally substituted alkylene chain; R⁴ is optionally substituted alkyl, optionally substituted aryl, optionally substituted aralkyl, optionally substituted heteroaryl or optionally substituted heteroarylalkyl; each R⁵ is independently hydrogen, alkyl, optionally substituted cycloalkyl, optionally substituted aryl or optionally substituted aralkyl; each R⁶ is alkyl, optionally substituted cycloalkyl, optionally substituted aralkyl or —R³C(O)OR⁴; or any R⁵ and R⁶, together with the nitrogen to which they are both attached, form an optionally substituted N-heterocyclyl or an optionally substituted N-heteroaryl; as a single stereoisomer or as a mixture thereof or a pharmaceutically acceptable salt thereof.

2. The method of claim 1, wherein the compound is 5-(tetradecyloxy)-2-furoic acid.

3. The method of claim 1 or claim 2, wherein the metabolic disorder is insulin resistance, hyperglycemia, type 2 diabetes mellitus, obesity, fatty liver disease, glucose intolerance, hyperinsulinemia, metabolic syndrome, or hypertension.

4. The method of any one of claims 1-3, wherein the metabolic disorder comprises insulin resistance.

5. The method of any one of claims 1-3, wherein the metabolic disorder comprises metabolic syndrome.

6. The method of any one of claims 1-3, wherein the metabolic disorder comprises type 2 diabetes mellitus.

7. The method of any one of claims 1-6, wherein the individual has a body mass index>30.0.

8. The method of any one of claims 1-7, wherein said administering results in a serum insulin level in a normal range.

9. The method of any one of claims 1-7, wherein said administering results in a blood glucose level in a normal range.

10. The method of any one of claims 1-9, further comprising administering at least one additional therapeutic agent.

11. The method of claim 10, wherein the at least one additional therapeutic agent is insulin, an insulin analog, a biguanidine, or a thiazolidinedione.

12. The method of any one of claims 1-11, wherein said administering is via oral administration.

13. The method of any one of claims 1-12, wherein the compound of Formula I is administered daily.

14. The method of any one of claims 1-12, wherein the compound of Formula I is administered once per week.

15. The method of any one of claims 1-12, wherein the compound of Formula I is administered via controlled delivery.

16. The method of claim 15, wherein the compound of Formula I is present in an implantable delivery device.

17. A method of treating metabolic syndrome in an individual, the method comprising administering to the individual an effective amount of a compound of Formula I: 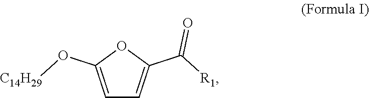
wherein: R¹ is R¹ is —O—R², —O—R³OR², OR³—OC(O)—N(R⁵)R⁶, —O—R³—N(R⁵)R⁶, —O—R³—N(R⁴)C(O)OR⁵, —O—R³—C(O)OR⁵, —O—R³—C(O)N(R⁵)R⁶or —N(R⁵)S(O)₂—R⁴; each R² is independently alkyl, haloalkyl, optionally substituted aryl, optionally substituted aralkyl, optionally substituted heterocyclyl, optionally substituted heterocyclylalkyl, optionally substituted heteroaryl or optionally substituted heteroarylalkyl; each R³ is independently an optionally substituted alkylene chain; R⁴ is optionally substituted alkyl, optionally substituted aryl, optionally substituted aralkyl, optionally substituted heteroaryl or optionally substituted heteroarylalkyl; each R⁵ is independently hydrogen, alkyl, optionally substituted cycloalkyl, optionally substituted aryl or optionally substituted aralkyl; each R⁶ is alkyl, optionally substituted cycloalkyl, optionally substituted aralkyl or —R³C(O)OR⁴ ; or any R⁵ and R⁶, together with the nitrogen to which they are both attached, form an optionally substituted N-heterocyclyl or an optionally substituted N-heteroaryl; as a single stereoisomer or as a mixture thereof or a pharmaceutically acceptable salt thereof.

18. The method of claim 17, wherein the compound is 5-(tetradecyloxy)-2-furoic acid.

19. The method of claim 17 or claim 18, wherein said administering is via oral administration.

20. The method of any one of claims 17-19, wherein the compound of Formula I is administered daily.

21. The method of any one of claims 17-19, wherein the compound of Formula I is administered once per week.

22. The method of any one of claims 17-19, wherein the compound of Formula I is administered via controlled delivery.

23. The method of claim 22, wherein the compound of Formula I is present in an implantable delivery device.
